(19) United States
(12) Patent Application Publication
(10) Pub. No.: US 2022/0096338 A1
(43) Pub. Date: Mar. 31, 2022

(54) COMPOSITIONS AND METHODS FOR STYLING HAIR

(71) Applicant: L'Oreal, Paris (FR)

(72) Inventors: Rita CHOKSHI, Monroe Township, NJ (US); Seyma ASLAN, Clifton, NJ (US); Emma NAIBERK, Maplewood, NJ (US); Daisuke ISHII, Paramus, NJ (US); Xian Zhi ZHOU, Milburn, NJ (US); Lisa Chuyin YE-TSE, Brooklyn, NY (US)

(21) Appl. No.: 17/489,196

(22) Filed: Sep. 29, 2021

Related U.S. Application Data
(60) Provisional application No. 63/086,020, filed on Sep. 30, 2020.

(30) Foreign Application Priority Data
Nov. 18, 2020 (FR) 2011812

(51) Int. Cl. A61K 8/04; A61Q 5/06; A61K 8/73; A61K 8/81; A61K 8/894

(52) U.S. Cl. CPC A61K 8/042; A61Q 5/06; A61K 8/737; A61K 8/73; A61K 2800/594; A61K 8/894; A61K 2800/5422; A61K 2800/596; A61K 8/8182

(57) ABSTRACT

The disclosure relates to hair styling and/or shaping compositions having a unique transformative nature that allows the composition to change from a gel to a foam when a shear stress is applied to the gel. The disclosure also relates to packaging systems comprising the compositions, and methods of using the compositions.

### TECHNICAL FIELD

The present disclosure relates to compositions for styling the hair, packaging systems comprising the compositions, and methods of using the compositions.

### BACKGROUND

Conventional hair styling compositions are typically in the forms of lotions, gels, mousses, creams, sprays, serums, wax, oils, clays, etc. It is understood that different forms of compositions can provide different benefits, yet can also present different drawbacks and/or challenges, such as, for example, when the particular composition is applied to hair.

For example, hair gels are a common form for styling compositions for curly hair because they are usually good at providing definition and fighting frizz. However, gels may have a tendency to be thick or have heavy oils mixed in. Thick gels may be too heavy for fine hair and may weigh down the curls of curly hair. In addition, gels may be difficult to apply evenly throughout the hair, and may lead to a “crunchy” sensation on the hair.

Foam compositions, such as mousses, are another form of hair styling composition. There are two types of foam hair styling compositions, aerosol type and non-aerosol type. The aerosol type compositions typically require a propellant and the compositions are usually filled in an aerosol pressure resistant container and are dispensed from the nozzle of the container in the form of a foam. The non-aerosol type hair styling composition does not use a propellant, but the composition is typically filled in a foamer container and produces foam when shear stress is applied, e.g. through a pump. Currently existing non-aerosol hair styling compositions transform a liquid composition to a foam.

As such, there is a need for hair styling compositions that can be easily applied to hair, give good curl definition, provide long-lasting hold, hair manageability, and good cosmetic properties, and/or be environmentally friendly. Ideally, new galenic forms of compositions are desired.

It has now surprisingly been found that compositions according to the disclosure have a unique transformative nature wherein the composition is provided in the form of a gel that can transform into a foam under application of shear stress. These foam compositions can be easy to apply, provide curl definition, and/or may impart various advantageous sensory properties to the hair.

### BRIEF DESCRIPTION OF FIGURES

The accompanying drawings, which are incorporated herein and constitute part of this specification, illustrate exemplary embodiments of the disclosure, and, together with the general description given above and the description provided herein, serve to explain features of the disclosure.

FIG. 1 is a graph illustrating a typical rheological property of a general gel.

FIG. 2 is an image illustrating an exemplary texture transformation of a gel-to-foam composition under shear stress, according to an embodiment of the disclosure.

FIG. 3 is a graph illustrating the properties of a gel-to-foam composition according to an embodiment of the disclosure, in comparison with four commercially available hair care products.

FIG. 4 is a set of images illustrating exemplary foams generated from a gel-to-foam composition according to an embodiment of the disclosure.

It is to be understood that the foregoing and following descriptions are exemplary and explanatory only, and are not intended to be restrictive of any subject matter claimed.

### SUMMARY

The disclosure relates to hair styling and/or shaping compositions which have a unique transformative nature such that the compositions can transform from a gel to a foam. Compositions disclosed herein can, in certain embodiments, impart various cosmetic properties to hair, such as one or more of styling/shaping, curl definition, frizz control, retention of shape/curl, curl pick up, discipline, hydration, moisture, shine, and conditioning. The disclosure also relates to packaging systems comprising the compositions, and methods of using the compositions.

In various embodiments, the disclosure relates to gel compositions comprising (a) at least one nonionic guar polymer; (b) at least one polysaccharide other than the at least one nonionic guar polymer; (c) at least one nonionic film forming polymer; (d) at least one nonionic surfactant; (e) at least one anionic surfactant; (f) at least one silicone compound; and (g) at least one polyol. In various embodiments, the at least one nonionic guar polymer comprises a nonionic hydroxyalkyl-modified guar polymer, such as hydroxypropyl guar. In further embodiments, the at least one polysaccharide other than the at least one nonionic guar polymer is chosen from gums, celluloses, starches, or mixtures thereof, for example xanthan gum. In still further embodiments, the weight ratio of the at least one polysaccharide other than the at least one nonionic guar polymer to the at least one nonionic guar polymer ranges from about 1:0.01 to about 1:1. In further embodiments the at least one nonionic film forming polymer is chosen from vinylpyrrolidone homopolymers, copolymers of vinylpyrrolidone and of vinyl acetate, polyalkyloxazolines, vinyl acetate homopolymers, copolymers of vinyl acetate and of acrylic ester, copolymers of vinyl acetate and of ethylene, copolymers of vinyl acetate and of maleic ester, copolymers of polyethylene and of maleic anhydride, alkyl acrylate homopolymers and alkyl methacrylate homopolymers, copolymers of acrylonitrile and of a non-ionic monomer, or a mixture thereof, for example VP/VA copolymer. In yet further embodiments, the at least one nonionic surfactant is chosen from alkyl or polyalkyl esters of poly(ethylene oxide), alkyl or polyalkyl ethers of poly(ethylene oxide), optionally polyoxyethylenated alkyl or polyalkyl esters of sorbitan, optionally polyoxyethylenated alkyl or polyalkyl ethers of sorbitan, alkyl or polyalkyl glycosides polyglycosides, alkyl or polyalkyl esters of sucrose, optionally polyoxyethylenated alkyl or polyalkyl esters of glycerol, salts thereof, or mixtures thereof, such as, for example, an alkylpolyglucoside. In even further embodiments, the at least one anionic surfactant is chosen from non-sulfate anionic surfactants comprising alkyl sulfonates, alkyl sulfosuccinates, alkyl sulfoacetates, acyl isethionates, alkoxylated monoacids, acyl amino acids, acyl glycinates, acyl glutamates, acyl sarcosinates, salts thereof, or mixtures thereof, such as sodium C₁₄₋₁₆ olefin sulfonate. In yet further embodiments, the at least one silicone compound is chosen from dimethicone copolyols. In certain embodiments, the at least one polyol is chosen from sugar alcohols and alkane polyols, and optionally, at the composition comprises two polyols, where one polyol is a sugar alcohol and one polyol is an alkane polyol. In yet further embodiments, the sugar alcohol is sorbitol and the alkane polyol is glycerin. The compositions may be in the form of a gel or a foam. The gel compositions have a transformative texture that can be transformed into a foam when a shear stress is applied to the gel by mechanical means.

In various embodiments, the disclosure relates to packaging systems comprising a dispensing container containing a gel composition, wherein the gel composition comprises (a) at least one nonionic guar polymer; (b) at least one polysaccharide other than the at least one nonionic guar polymer; (c) at least one nonionic film forming polymer; (d) at least one nonionic surfactant; (e) at least one anionic surfactant; (f) at least one silicone compound; and (g) at least one polyol, wherein the dispensing container comprises a non-aerosol dispensing device configured to provide shear stress to the gel composition sufficient to transform the form of the composition into a foam. In various embodiments, the at least one nonionic guar polymer comprises a nonionic hydroxyalkyl-modified guar polymer, such as hydroxypropyl guar. In further embodiments, the at least one polysaccharide other than the at least one nonionic guar polymer is chosen from gums, celluloses, starches, or mixtures thereof, for example xanthan gum. In still further embodiments, the weight ratio of the at least one polysaccharide other than the at least one nonionic guar polymer to the at least one nonionic guar polymer ranges from about 1:0.01 to about 1:1. In further embodiments the at least one nonionic film forming polymer is chosen from vinylpyrrolidone homopolymers, copolymers of vinylpyrrolidone and of vinyl acetate, polyalkyloxazolines, vinyl acetate homopolymers, copolymers of vinyl acetate and of acrylic ester, copolymers of vinyl acetate and of ethylene, copolymers of vinyl acetate and of maleic ester, copolymers of polyethylene and of maleic anhydride, alkyl acrylate homopolymers and alkyl methacrylate homopolymers, copolymers of acrylonitrile and of a non-ionic monomer, or a mixture thereof, for example VP/VA copolymer. In yet further embodiments, the at least one nonionic surfactant is chosen from alkyl or polyalkyl esters of poly(ethylene oxide), alkyl or polyalkyl ethers of poly(ethylene oxide), optionally polyoxyethylenated alkyl or polyalkyl esters of sorbitan, optionally polyoxyethylenated alkyl or polyalkyl ethers of sorbitan, alkyl or polyalkyl glycosides polyglycosides, alkyl or polyalkyl esters of sucrose, optionally polyoxyethylenated alkyl or polyalkyl esters of glycerol, salts thereof, or mixtures thereof, such as, for example, an alkylpolyglucoside. In even further embodiments, the at least one anionic surfactant is chosen from non-sulfate anionic surfactants comprising alkyl sulfonates, alkyl sulfosuccinates, alkyl sulfoacetates, acyl isethionates, alkoxylated monoacids, acyl amino acids, acyl glycinates, acyl glutamates, acyl sarcosinates, salts thereof, or mixtures thereof, such as sodium C₁₄₋₁₆ olefin sulfonate. In yet further embodiments, the at least one silicone compound is chosen from dimethicone copolyols. In certain embodiments, the at least one polyol is chosen from sugar alcohols and alkane polyols, and optionally, at the composition comprises two polyols, where one polyol is a sugar alcohol and one polyol is an alkane polyol.

In various embodiments, the disclosure relates to methods for styling hair, the method comprising (i) applying shear stress to a gel composition comprising (a) at least one nonionic guar polymer; (b) at least one polysaccharide other than the at least one nonionic guar polymer; (c) at least one nonionic film forming polymer; (d) at least one nonionic surfactant; (e) at least one anionic surfactant; (f) at least one silicone compound; and (g) at least one polyol, wherein after the shear stress is applied to the gel composition, the composition is in the form of a foam; and (ii) applying the foam to the hair. In various embodiments, the at least one nonionic guar polymer comprises a nonionic hydroxyalkyl-modified guar polymer, such as hydroxypropyl guar. In further embodiments, the at least one polysaccharide other than the at least one nonionic guar polymer is chosen from gums, celluloses, starches, or mixtures thereof, for example xanthan gum. In still further embodiments, the weight ratio of the at least one polysaccharide other than the at least one nonionic guar polymer to the at least one nonionic guar polymer ranges from about 1:0.01 to about 1:1. In further embodiments the at least one nonionic film forming polymer is chosen from vinylpyrrolidone homopolymers, copolymers of vinylpyrrolidone and of vinyl acetate, polyalkyloxazolines, vinyl acetate homopolymers, copolymers of vinyl acetate and of acrylic ester, copolymers of vinyl acetate and of ethylene, copolymers of vinyl acetate and of maleic ester, copolymers of polyethylene and of maleic anhydride, alkyl acrylate homopolymers and alkyl methacrylate homopolymers, copolymers of acrylonitrile and of a non-ionic monomer, or a mixture thereof, for example VP/VA copolymer. In yet further embodiments, the at least one nonionic surfactant is chosen from alkyl or polyalkyl esters of poly(ethylene oxide), alkyl or polyalkyl ethers of poly(ethylene oxide), optionally polyoxyethylenated alkyl or polyalkyl esters of sorbitan, optionally polyoxyethylenated alkyl or polyalkyl ethers of sorbitan, alkyl or polyalkyl glycosides polyglycosides, alkyl or polyalkyl esters of sucrose, optionally polyoxyethylenated alkyl or polyalkyl esters of glycerol, salts thereof, or mixtures thereof, such as, for example, an alkylpolyglucoside. In even further embodiments, the at least one anionic surfactant is chosen from non-sulfate anionic surfactants comprising alkyl sulfonates, alkyl sulfosuccinates, alkyl sulfoacetates, acyl isethionates, alkoxylated monoacids, acyl amino acids, acyl glycinates, acyl glutamates, acyl sarcosinates, salts thereof, or mixtures thereof, such as sodium C₁₄₋₁₆ olefin sulfonate. In yet further embodiments, the at least one silicone compound is chosen from dimethicone copolyols. In certain embodiments, the at least one polyol is chosen from sugar alcohols and alkane polyols, and optionally, at the composition comprises two polyols, where one polyol is a sugar alcohol and one polyol is an alkane polyol.

In one embodiment, the disclosure relates to a gel composition comprising (a) at least one hydroxyalkyl guar; (b) at least one polysaccharide other than the at least one nonionic guar polymer chosen from gums; (c) at least one nonionic film forming polymer chosen from vinylpyrrolidone homopolymers and copolymers of vinylpyrrolidone and of vinyl acetate; (d) at least one nonionic surfactant chosen from alkylpolyglucosides; (e) at least one anionic surfactant chosen from alkyl sulfonates and their salts; (f) at least one dimethicone copolyol; and (g) at least one polyol chosen from sugar alcohols and at least one polyol chosen from alkane polyols, wherein the gel composition has a transformative texture that can be transformed into a foam when a shear stress is applied. In various embodiments, the (a) at least one hydroxyalkyl guar is present in an amount ranging from about 0.1% to about 0.5%; (b) at least one polysaccharide other than the at least one nonionic guar polymer chosen from gums is present in an amount ranging from about 0.5% to about 1.5%; (c) at least one nonionic film forming polymer chosen from copolymers of vinylpyrrolidone and of vinyl acetate is present in an amount ranging from about 1% to about 6%; (d) at least one nonionic surfactant chosen from alkylpolyglucosides is present in an amount ranging from about 0.5% to about 4%; (e) at least one anionic surfactant chosen from alkyl sulfonates and their salts is present in an amount ranging from about 0.1% to about 2%; (f) at least one dimethicone copolyol is present in an amount ranging from about 0.5% to about 4%; and (g) at least one polyol chosen from sugar alcohols is present in an amount ranging from about 0.1% to about 2%, and at least one polyol chosen from alkane polyols is present in an amount ranging from about 0.5% to about 8%. The weight ratio of at least one polysaccharide other than the at least one nonionic guar polymer chosen from gums to the at least one at least one hydroxyalkyl guar may be about 1:0.25 or about 0.9:0.25.

### DETAILED DESCRIPTION

The disclosure relates to compositions for styling and/or shaping hair, packaging systems comprising the compositions, and methods of using the compositions.

The hair styling compositions according to the disclosure have unique textures of gels that can transform into foams. Typical gel compositions are better in consistency and stronger at hold, while typical foam compositions are easier to spread and have better coverage. Thus, compositions according to the disclosure provide the benefits of both a gel and a foam.

I. Compositions

In exemplary and non-limiting embodiments, compositions according to the disclosure comprise (a) at least one nonionic guar polymer; (b) at least one polysaccharide other than the at least one nonionic guar polymer; (c) at least one nonionic film forming polymer; (d) at least one nonionic surfactant; (e) at least one anionic surfactant; (f) at least one silicone copolymer; and (g) at least one polyol.

A propellant is not required for the compositions. As such, the compositions may be free or essentially free of propellant. The compositions may be further free or essentially free of sulfate-based anionic surfactants and/or cationic film forming polymers.

### Nonionic Guar Polymer

Compositions according to the disclosure comprise at least one nonionic guar polymer. Non-limiting examples of nonionic guar polymers that can be used include unmodified and modified guar polymers. For example, modified guar polymers include those that are modified by hydroxyalkyl groups, for example C1-C6 hydroxyalkyl groups. Non-limiting examples of hydroxyalkyl groups include hydroxymethyl, hydroxyethyl, hydroxypropyl, and hydroxybutyl groups. For example, in one exemplary embodiment, the at least one nonionic guar polymer is chosen from hydroxypropyl guar.

Non-limiting examples of nonionic, unmodified guar gums that may be used in various embodiments include Guargel D/15 (Noveon); Vidogum GH 175 (Unipectine), Meypro-Guar 50 and JAGUAR® C (Meyhall/Rhodia Chimie). Non-limiting examples of nonionic modified guar gums include Jaguar® HP8, HP60, HP120, DC 293 and HP 105 (Meyhall/Rhodia Chimie); Galactasol 4H4FD2 (Ashland); and N-Hance® HP by the company Aqualon.

In various exemplary embodiments, the total amount of the at least one nonionic guar polymer may vary, but typically ranges from about 0.01% to about 2%, including all subranges therebetween, such as from about 0.01% to about 1.9%, from about 0.01% to about 1.8%, from about 0.01% to about 1.7%, from about 0.01% to about 1.6%, from about 0.01% to about 1.5%, from about 0.01% to about 1.4%, from about 0.01% to about 1.3%, from about 0.01% to about 1.2%, from about 0.01% to about 1.1%, from about 0.01% to about 1%, from about 0.01% to about 0.9%, from about 0.01% to about 0.8%, from about 0.01% to about 0.7%, from about 0.01% to about 0.6%, from about 0.01% to about 0.5%, from about 0.01% to about 0.4%, from about 0.01% to about 0.3%, from about 0.01% to about 0.2%, from about 0.01% to about 0.1%, from about 0.1% to about 2%, from about 0.1% to about 1.9%, from about 0.1% to about 1.8%, from about 0.1% to about 1.7%, from about 0.1% to about 1.6%, from about 0.1% to about 1.5%, from about 0.1% to about 1.4%, from about 0.1% to about 1.3%, from about 0.1% to about 1.2%, from about 0.1% to about 1.1%, from about 0.1% to about 1%, from about 0.1% to about 0.9%, from about 0.1% to about 0.8%, from about 0.1% to about 0.7%, from about 0.1% to about 0.6%, from about 0.1% to about 0.5%, from about 0.1% to about 0.4%, from about 0.1% to about 0.3%, from about 0.1% to about 0.2%, from about 0.2% to about 2%, from about 0.2% to about 1.9%, from about 0.2% to about 1.8%, from about 0.2% to about 1.7%, from about 0.2% to about 1.6%, from about 0.2% to about 1.5%, from about 0.2% to about 1.4%, from about 0.2% to about 1.3%, from about 0.2% to about 1.2%, from about 0.2% to about 1%, from about 0.2% to about 0.9%, from about 0.2% to about 0.8%, from about 0.2% to about 0.7%, from about 0.2% to about 0.6%, from about 0.2% to about 0.5%, from about 0.2% to about 0.4%, or from about 0.2% to about 0.3% by weight, relative to the total weight of the composition.

### Polysaccharide

The compositions further comprise at least one polysaccharide other than the at least one nonionic guar polymer. Polysaccharides are polymers which exhibit monosaccharides or disaccharides as base units. The polysaccharides other than the at least one nonionic guar polymer which can be used in the compositions according to the present invention include, by way of example only, gums, celluloses, and starches.

Non-limiting examples of gums include acacia, agar, algin, alginic acid, ammonium alginate, amylopectin, calcium alginate, calcium carrageenan, carnitine, carrageenan, dextrin, gelatin, gellan gum, guar gum, hectorite, hyaluronic acid, hydrated silica, hydroxypropyl chitosan, hydroxypropyl guar, karaya gum, kelp, locust bean gum, natto gum, potassium alginate, potassium carrageenan, propylene glycol alginate, sclerotium gum, sodium carboxymethyl dextran, sodium carrageenan, tragacanth gum, xanthan gum, and biosacharide gum. Modified gums or derivatives of gums may also be used, such as, for example, deacylated gellan gum, welan gum, or hydroxypropylated guar gum, such as Jaguar HP 105 sold by Rhodia.

Non-limiting examples of celluloses include hydroxyalkylcelluloses, such as hydroxyethylcelluloses, hydroxypropylmethylcellulose, or hydropropylcelluloses, which may or may not contain a fatty chain. One particularly suitable hydroxypropylmethylcellulose is Methocel F4M sold by Dow Chemicals (INCI name: hydroxypropylmethylcellulose). Celluloses modified with groups comprising one or more nonionic fatty chains that can be used include hydroxyethylcelluloses, preferably nonionic hydroxyethylcelluloses, modified by groups comprising at least one fatty chain, such as alkyl, arylalkyl or alkylaryl groups, or their mixtures, and in which the alkyl groups are preferably C8-C22 alkyl groups, such as the product NATROSOL™ Plus Grade 330 CS (C16 alkyls), sold by Aqualon, corresponding to the INCI name cetylhydroxyethylcellulose, or the product BERMOCOLL® EHM 100 sold by Berol Nobel, and those modified with alkylphenyl polyalkylene glycol ether groups, such as the product AMERCELL POLYMER® HM-1500 (nonylphenyl polyethylene glycol (15) ether) sold by Amerchol that corresponds to the INCI name nonoxynyl hydroxyethylcellulose.

Non-limiting examples of starches include modified starches, starch-based polymers, methylhydroxypropyl starch, potato starch, wheat starch, rice starch, starch crosslinked with octenyl succinic anhydride, starch oxide, dialdehyde starch, dextrin, British gum, acetyl starch, starch phosphate, carboxymethyl starch, hydroxyethyl starch, and hydroxypropyl starch.

In various exemplary embodiments, the total amount of the one or more polysaccharide other than the nonionic guar polymer may vary, but is typically ranges from about 0.01% to about 2%, including all subranges therebetween, such as from about 0.01% to about 1.9%, from about 0.01% to about 1.8%, from about 0.01% to about 1.7%, from about 0.01% to about 1.6%, from about 0.01% to about 1.5%, from about 0.01% to about 1.4%, from about 0.01% to about 1.3%, from about 0.01% to about 1.2%, from about 0.01% to about 1.1%, from about 0.01% to about 1%, from about 0.01% to about 0.9%, from about 0.01% to about 0.8%, from about 0.01% to about 0.7%, from about 0.01% to about 0.6%, from about 0.01% to about 0.5%, from about 0.1% to about 2%, from about 0.1% to about 1.9%, from about 0.1% to about 1.8%, from about 0.1% to about 1.7%, from about 0.1% to about 1.6%, from about 0.1% to about 1.5%, from about 0.1% to about 1.4%, from about 0.1% to about 1.3%, from about 0.1% to about 1.2%, from about 0.1% to about 1.1%, from about 0.1% to about 1%, from about 0.1% to about 0.9%, from about 0.1% to about 0.8%, from about 0.1% to about 0.7%, from about 0.1% to about 0.6%, from about 0.1% to about 0.5%, from about 0.2% to about 2%, from about 0.2% to about 1.9%, from about 0.2% to about 1.8%, from about 0.2% to about 1.7%, from about 0.2% to about 1.6%, from about 0.2% to about 1.5%, from about 0.2% to about 1.4%, from about 0.2% to about 1.3%, from about 0.2% to about 1.2%, from about 0.2% to about 1.1%, from about 0.2% to about 1%, from about 0.2% to about 0.9%, from about 0.2% to about 0.8%, from about 0.2% to about 0.7%, from about 0.2% to about 0.6%, or from about 0.2% to about 0.5% by weight, relative to the total weight of the composition. In at least certain embodiments, the composition comprises a polysaccharide other than the nonionic guar polymer in an amount less than about 2%, such as less than about 1.75%, less than about 1.5%, less than about 1.25%, or less than about 1% by weight, relative to the total weight of the composition. In other embodiments, the at least one polysaccharide other than the nonionic guar polymer in compositions according to the disclosure is present in an amount ranging from about 0.6% to about 1.2% by weight, relative to the total weight of the composition. In at least one embodiment, the at least one polysaccharide other than the nonionic guar polymer includes xanthan gum.

In various exemplary embodiments, the weight ratio of the at least one polysaccharide other than the nonionic guar polymer to the at least one nonionic guar polymer ranges from about 1:0.01 to about 1:1, such as for example ranging from 1:0.05 to about 1:1, from about 1:0.1 to about 1:0.8, from about 1:0.25 to about 1:0.8, or from about 1:0.5 to about 1:0.25. In at least one exemplary embodiment, the weight ratio of at least one polysaccharide other than the nonionic guar polymer to the at least one nonionic guar polymer is about 1:0.25 or about 0.9:0.25 (i.e., about 1:0.28).

### Nonionic Film Forming Polymers

Compositions according to the present disclosure further comprise at least one nonionic film forming polymer. As used herein, a film forming polymer is meant to include a polymer that is capable, by itself or in the presence of an auxiliary film forming agent, of forming a substantially continuous film that adheres to keratin materials, and preferably a substantially cohesive film.

Non-limiting examples of nonionic film-forming polymers include vinylpyrrolidone homopolymers (also known as PVP); copolymers of vinylpyrrolidone and of vinyl acetate; polyalkyloxazolines; vinyl acetate homopolymers; copolymers of vinyl acetate and of acrylic ester; copolymers of vinyl acetate and of ethylene; copolymers of vinyl acetate and of maleic ester; copolymers of polyethylene and of maleic anhydride; alkyl acrylate homopolymers and alkyl methacrylate homopolymers; copolymers of acrylonitrile and of a non-ionic monomer; and a mixture thereof. In some cases, particularly useful nonionic film-forming polymers include vinylpyrrolidone homopolymers and copolymers of vinylpyrrolidone and of vinyl acetate, for example, polyvinylpyrrolidone/vinyl acetate (VP/VA) copolymer.

By way of example, the nonionic film-forming polymers may be chosen from vinylpyrrolidone homopolymers; copolymers of vinylpyrrolidone and of vinyl acetate; polyalkyloxazolines, such as the polyethyloxazolines provided by the company Polymer Chemistry Innovations under the names Aquazol HP, and Aquzol HVIS; vinyl acetate homopolymers, such as the product provided under the name UCAR 130 Latex Resin by the company Dow Chemical or the product provided under the name Ultrapure Polymer 2041-R 012 by the company Ultra Chemical, Inc.; copolymers of vinyl acetate and of acrylic ester, such as the product provided under the name Rhodopas AD 310 from Rhone-Poulenc; copolymers of vinyl acetate and of ethylene, such as the product provided under the name Dermacryl LOR by the company Akzo Nobel; copolymers of vinyl acetate and of maleic ester, for example of dibutyl maleate, such as the product provided under the name Appretan MB Extra by the company Clariant; copolymers of polyethylene and of maleic anhydride; alkyl acrylate homopolymers and alkyl methacrylate homopolymers, such as the product provided under the name Micropearl RQ 750 by the company Matsumoto or the product provided under the name Luhydran® A 848 S by the company BASF; copolymers of acrylonitrile and of a non-ionic monomer chosen, for example, from butadiene and alkyl (meth)acrylates; mention may be made of the products provided under the names Nipol LX 531 B by the company Nippon Zeon or those provided under the name CJ 0601 B by the company Rohm and Haas; polyurethanes, such as the products provided under the names Acrysol RM 1020 or Acrysol RM 2020 by the company Dow Chemical or the products Uraflex XP 401 UZ or Uraflex XP 402 UZ by the company DSM Resins; copolymers of alkyl acrylate and of urethane, such as the product 8538-33 by the company National Starch; and polyam ides, such as the product Estapor LO 11 provided by the company Rhone-Poulenc.

In one embodiment, the at least one nonionic film forming polymer comprise only VP/VA copolymer (or PVP/VA copolymer). VP/VA copolymer has been found to be compatible with the other ingredients disclosed herein and, together with the other ingredients, gives the composition an appearance of semi clear or clear gel when shear stress is not applied to the composition. In further embodiments, the at least one non-ionic film forming polymer comprises PVP. In yet further embodiments, the at least one non-ionic film forming polymer is chosen from VP/VA copolymer, PVP, or a mixture thereof.

In various exemplary embodiments, the amount of the at least one the at least one nonionic film forming polymer may vary, but typically ranges from about 0.1% to about 15%, including all subranges therebetween, such as from about 0.1% to about 12%, from about 0.1% to about 10%, from about 0.1% to about 9%, from about 0.1% to about 8%, from about 0.1% to about 7%, from about 0.1% to about 6%, from about 0.1% to about 5%, from about 0.1% to about 4%, from about 0.5% to about 15%, from about 0.5% to about 12%, from about 0.5% to about 10%, from about 0.5% to about 9%, from about 0.5% to about 8%, from about 0.5% to about 7%, from about 0.5% to about 6%, from about 0.5% to about 5%, from about 0.5% to about 4%, from about 1% to about 15%, from about 1% to about 12%, from about 1% to about 10%, from about 1% to about 9%, from about 1% to about 8%, from about 1% to about 7%, from about 1% to about 6%, from about 1% to about 5%, from about 1% to about 4%, from about 1.5% to about 15%, from about 1.5% to about 12%, from about 1.5% to about 10%, from about 1.5% to about 9%, from about 1.5% to about 8%, from about 1.5% to about 7%, from about 1.5% to about 6%, from about 1.5% to about 5%, from about 1.5% to about 4%, from about 2% to about 15%, from about 2% to about 12%, from about 2% to about 10%, from about 2% to about 9%, from about 2% to about 8%, from about 2% to about 7%, from about 2% to about 6%, from about 2% to about 5%, or from about 2% to about 4% by weight, relative to the total weight of the composition. In at least certain embodiments, the at least one nonionic film forming polymer is in an amount ranges from about 2% to about 5% by weight, such as about 2.5% to about 4.5%, relative to the total weight of the composition.

### Nonionic Surfactants

Compositions according to the disclosure comprise at least one nonionic surfactant. The at least one nonionic surfactant may optionally be derived from plants. In certain embodiments, the nonionic surfactant may be chosen from alkyl and polyalkyl esters of poly(ethylene oxide), alkyl and polyalkyl ethers of poly(ethylene oxide), optionally polyoxyethylenated alkyl and polyalkyl esters of sorbitan, optionally polyoxyethylenated alkyl and polyalkyl ethers of sorbitan, alkyl and polyalkyl glycosides or polyglycosides, for example alkyl and polyalkyl glucosides or polyglucosides, alkyl and polyalkyl esters of sucrose, optionally polyoxyethylenated alkyl and polyalkyl esters of glycerol, and optionally polyoxyethylenated alkyl and polyalkyl ethers of glycerol, and mixtures thereof.

Exemplary and nonlimiting alkyl and polyalkyl esters of poly(ethylene oxide) include those containing at least one C8-C30 alkyl radical, with a number of ethylene oxide (EO) units ranging from 2 to 200. Mention may be made, for example, of PEG-20 stearate, PEG-40 stearate, PEG-100 stearate, PEG-20 laurate, PEG-8 laurate, PEG-40 laurate, PEG-150 distearate, PEG-7 cocoate, PEG-9 cococate, PEG-8 oleate, PEG-10 oleate and PEG-40 hydrogenated castor oil.

Exemplary and nonlimiting alkyl and polyalkyl ethers of poly(ethylene oxide) include those containing at least one C8-C30 alkyl radical, with a number of ethylene oxide (EO) units ranging from 3 to 200. Mention may be made, for example, of laureth-3, laureth-4, laureth-7, laureth-23, ceteth-5, ceteth-7, ceteth-15, ceteth-23, oleth-5, oleth-7, oleth-10, oleth-12, oleth-20, oleth-50, phytosterol 30 EO, steareth-6, steareth-20, steareth-21, steareth-40, steareth-100, beheneth 100, ceteareth-7, ceteareth-10, ceteareth-15, ceteareth-25, pareth-3, pareth-23, C12-15 pareth-3, C12-13 pareth-4, C12-13 pareth-23, trideceth-3, trideceth-4, trideceth-5, trideceth-6, trideceth-7 and trideceth-10, and mixtures thereof.

Exemplary and nonlimiting polyoxyethylenated alkyl and polyalkyl esters of sorbitan include those with a number of ethylene oxide (EO) units ranging from 0 to 100. Mention may be made, for example, of sorbitan laurate, sorbitan laurate 4 EO, sorbitan laurate 20 EO (polysorbate 20), sorbitan palmitate 20 EO (polysorbate 40), sorbitan stearate 20 EO (polysorbate 60), sorbitan oleate 20 EO (polysorbate 80) and sorbitan trioleate 20 EO (polysorbate 85).

Exemplary and nonlimiting polyoxyethylenated alkyl and polyalkyl ethers of sorbitan include those with a number of ethylene oxide (EO) units ranging from 0 to 100.

Exemplary and nonlimiting alkyl and polyalkyl glucosides or polyglucosides include those containing an alkyl group comprising from 6 to 30 carbon atoms and preferably from 6 to 18 or even from 8 to 16 carbon atoms, and containing a glucoside group preferably comprising from 1 to 5 and especially 1, 2, or 3 glucoside units.

In certain embodiments, exemplary and useful alkyl polyglucosides include those having the following formula (I):

R¹—O—(R²O)ₙ—Z(ₓ) (I)

wherein:
R¹ is an alkyl group having 8-18 carbon atoms;
R² is an ethylene or propylene group;
Z is a saccharide group with 5-6 carbon atoms;
n is an integer ranging from 0 to 10; and
x is an integer ranging from 1 to 5

The alkylpolyglucosides may be chosen, for example, from decylglucoside, for instance the product sold under the name MYDOL10® by the company Kao Chemicals or the product sold under the name PLANTACARE® 2000 UP by the company BASF and the product sold under the name ORAMIX™ NS 10 by the company SEPPIC; caprylyl/capryl glucoside, for instance the product sold under the name PLANTACARE® KE 3711 by the company Cognis or ORAMIX™ CG 110 by the company SEPPIC; laurylglucoside, for instance the product sold under the name PLANTACARE® 1200 UP by the company BASF or PLANTAREN 1200 N® by the company BASF; cocoglucoside, for instance the product sold under the name PLANTACARE® 818 UP by the company BASF; caprylylglucoside, for instance the product sold under the name PLANTACARE® 810 UP by the company BASF, octyl glucoside, or mixtures thereof.

Exemplary and nonlimiting alkyl and polyalkyl esters of sucrose that may be mentioned are Crodesta™ F150, sucrose monolaurate sold under the name Crodesta SL 40, and the products sold by Ryoto Sugar Ester, for instance sucrose palm itate sold under the reference Ryoto™ Sugar Ester P1670, Ryoto™ Sugar Ester LWA 1695 or Ryoto Sugar™ Ester 01570. Sucrose monooleate, monomyristate, and monostearate are also suitable for use.

Exemplary and nonlimiting (poly)oxyethylenated alkyl and polyalkyl esters of glycerol include those with a number of ethylene oxide (EO) units ranging from 0 to 100 and a number of glycerol units ranging from 1 to 30. Mention may be made, for example, of hexaglyceryl monolaurate, PEG-30 glyceryl stearate, polyglyceryl-2 laurate, polyglyceryl-10 laurate, polyglyceryl-10 stearate, polyglyceryl-10 oleate, PEG-7 glyceryl cocoate and PEG-20 glyceryl isostearate.

Exemplary and nonlimiting (poly)oxyethylenated alkyl and polyalkyl ethers of glycerol include those with a number of ethylene oxide (EO) units ranging from 0 to 100 and a number of glycerol units ranging from 1 to 30. Examples that may be mentioned include Nikkol Batyl Alcohol 100 and Nikkol Chimyl Alcohol 100.

In various exemplary embodiments, the total amount of the at least one nonionic surfactant may range from about 0.01% to about 10%, based on the total weight of the composition, including all ranges and subranges therebetween. For example, the total amount of the at least one nonionic surfactant may range from about 0.01% to about 8%, from about 0.01% to about 5%, from about 0.01% to about 4%, from about 0.01% to about 3%, from about 0.01% to about 2%, from about 0.1% to about 10%, from about 0.1% to about 8%, from about 0.1% to about 5%, from about 0.1% to about 4%, from about 0.1% to about 3%, from about 0.1% to about 2%, from about 0.5% to about 10%, from about 0.5% to about 8%, from about 0.5% to about 5%, from about 0.5% to about 4%, from about 0.5% to about 3%, from about 0.5% to about 2%, from about 0.75% to about 10%, from about 0.75% to about 8%, from about 0.75% to about 5%, from about 0.75% to about 4%, from about 0.75% to about 3%, or from about 0.75% to about 2%, by weight, relative to the total weight of the composition. In some embodiments, the at least one nonionic surfactant is present in an amount ranging from about 0.75% to about 3%, such as from about 1% to about 2%, or about 1.25% to about 1.75% by weight, based on the total weight of the composition.

### Anionic Surfactants

Compositions according to the disclosure comprise at least one anionic surfactant. In various embodiments, anionic surfactants included in the compositions disclosed herein are non-sulfate anionic surfactants.

Useful non-sulfate anionic surfactants include, but are not limited to, alkyl sulfonates, alkyl sulfosuccinates, alkyl sulfoacetates, acyl isethionates, alkoxylated monoacids, acyl amino acids such as acyl taurates, acyl glycinates, acyl glutamates, acyl sarcosinates, salts thereof, or mixtures thereof. Non-limiting examples of these non-sulfate anionic surfactants are provided below.

### Acyl Isethionates

Non-limiting examples of useful acyl isethionates and their salts include those of formula (IIa) and (IIb):

RCOOCHR¹CHR²X⁻M⁺ (IIa)

RCOOCHR¹CHR²X⁻Na⁺(IIb)

wherein:
R, R¹, and R² are each independently chosen from H or an alkyl chain having 1-24 carbon atoms, said chain being saturated or unsaturated, linear or branched;
X is COO⁻ or SO₃⁻; and
M is any suitable cation.

Although the cation may be chosen from any suitable cation including, for example, alkali metal ion such as sodium or potassium, ammonium ions, or alkanolammonium ions such as monoethanolammonium or triethanolammonium ions, sodium is a preferred cation. In various embodiments, RCO— represents the coconut acid moiety. Non-limiting examples of acyl isethionates include sodium cocoyl isethionate, sodium lauroyl isethionate, sodium lauroyl methyl isethionate, and sodium cocoyl methyl isethionate.

### Acyl Sarcosinates

Non-limiting examples of acyl sarcosinates and their salts include potassium lauroyl sarcosinate, potassium cocoyl sarcosinate, sodium cocoyl sarcosinate, sodium lauroyl sarcosinate, sodium myristoyl sarcosinate, sodium oleoyl sarcosinate, sodium palmitoyl sarcosinate, and ammonium lauroyl sarcosinate.

### Alkyl Sulfonates

Useful alkyl sulfonates and their salts include alkyl aryl sulfonates, primary alkane disulfonates, alkene sulfonates, hydroxyalkane sulfonates, alkyl glyceryl ether sulfonates, sulfonates of alkylphenolpolyglycol ethers, alkylbenzenesulfonates, phenylalkanesulfonates, alpha-olefinsulfonates, olefin sulfonates, alkene sulfonates, hydroxyalkanesulfonates and disulfonates, secondary alkanesulfonates, paraffin sulfonates, ester sulfonates, sulfonated fatty acid glycerol esters, and alpha-sulfo fatty acid methyl esters including methyl ester sulfonate.

In some instances, an alkyl sulfonate of formula (III) is particularly useful:

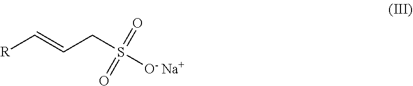

wherein R is selected from H or alkyl chain that has 1-24 carbon atoms, preferably 6-24 carbon atoms, more preferably, 8 to 20 carbon atoms, said chain being saturated or unsaturated, linear or branched, substituted or unsubstituted. Sodium is shown as the cation in the above formula (III) but the cation may be may be chosen from any suitable cation including, for example, alkali metal ion such as sodium or potassium, ammonium ions, or alkanolammonium ions such as monoethanolammonium or triethanolammonium ions.

In some instances, the alkyl sulfonate(s) are selected from C8-C16 alkyl benzene sulfonates, C10-C20 paraffin sulfonates, C₁₀-C₂₄ olefin sulfonates, salts thereof, or mixtures thereof. In certain embodiments, C₁₀-C₂₄ olefin sulfonates and salts thereof may be preferred. A non-limiting example of a C₁₀-C₂₄ olefin sulfonate that can be used is sodium C₁₄₋₁₆ olefin sulfonate.

### Alkyl Sulfosuccinates

Non-limiting examples of useful alkyl sulfosuccinates and their salts include those of formula (IV):

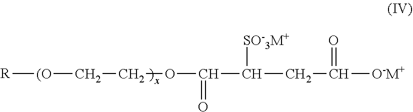

wherein:
R is a straight or branched chain alkyl or alkenyl group having 10 to 22 carbon atoms, preferably 10 to 20 carbon atoms;
x is a number that represents the average degree of ethoxylation, and can range from 0 to about 5, preferably from 0 to about 4, and most preferably from about 2 to about 3.5; and
M, which can be the same or different, is chosen from any suitable monovalent cation.

In some embodiments, cations are alkali metal ions such as sodium or potassium, ammonium ions, or alkanolammonium ions such as monoethanolammonium or triethanolammonium ions.

Non-limiting examples of alkyl sulfosuccinates salts include disodium oleamido MIPA sulfosuccinate, disodium oleamido MEA sulfosuccinate, disodium lauryl sulfosuccinate, disodium laureth sulfosuccinate, diammonium lauryl sulfosuccinate, diammonium laureth sulfosuccinate, dioctyl sodium sulfosuccinate, disodium oleamide MEA sulfosuccinate, sodium dialkyl sulfosuccinate, or mixtures thereof.

### Alkyl Sulfoacetates

Non-limiting examples of alkyl sulfoacetates and their salts include, for example, alkyl sulfoacetates such as C4-C18 fatty alcohol sulfoacetates and/or salts thereof. In some embodiments, a sulfoacetate salt is sodium lauryl sulfoacetate. Useful cations for the salts include alkali metal ions such as sodium or potassium, ammonium ions, or alkanolammonium ions such as monoethanolammonium or triethanolammonium ions.

### Alkoxylated Monoacids

Non-limiting examples of alkoxylated monoacids include compounds corresponding to formula (V):

R—O[CH₂O]ᵤ[(CH₂)ₓCH(R′)(CH₂)_{y}(CH₂)_{z}O]ᵥ[CH₂CH₂O]_{w}CH₂COOH (V)

wherein:

R is a hydrocarbon radical containing from about 6 to about 40 carbon atoms;

R′ represents hydrogen or alkyl;

u, v, and w, which may be identical or different, independently represent numbers from 0 to 60;

x, y, and z, which may be identical or different, independently represent numbers from 0 to 13; and

the sum of x+y+z>0.

Compounds corresponding to formula (V) can be obtained by alkoxylation of alcohols R—OH with ethylene oxide as the sole alkoxide or with several alkoxides and subsequent oxidation. The numbers u, v, and w each represent the degree of alkoxylation. Whereas, on a molecular level, the numbers u, v, and w and the total degree of alkoxylation can only be integers, including zero, on a macroscopic level they are mean values in the form of broken numbers.

In formula (V), R is linear or branched, acyclic or cyclic, saturated or unsaturated, aliphatic or aromatic, substituted or unsubstituted. For example, R may be a linear or branched, acyclic C6-C40 alkyl or alkenyl group or a C1-C40 alkyl phenyl group, more typically a C8-C22 alkyl or alkenyl group, or a C4-C18 alkyl phenyl group, and even more typically a C12-C18 alkyl group or alkenyl group or a C6-C16 alkyl phenyl group. Further, u, v, w, independently of one another, may be chosen from a number ranging from 2 to 20, such as a number ranging from 3 to 17, or a number ranging from 5 to 15. Further still, x, y, z, independently of one another, may be chosen from a number ranging from 0 to 13, such as a number ranging from 1 to 10, or a number ranging from 2 to 8.

Suitable alkoxylated monoacids include, but are not limited to: Butoxynol-5 Carboxylic Acid, Butoxynol-19 Carboxylic Acid, Capryleth-4 Carboxylic Acid, Capryleth-6 Carboxylic Acid, Capryleth-9 Carboxylic Acid, Ceteareth-25 Carboxylic Acid, Coceth-7 Carboxylic Acid, C9-11 Pareth-6 Carboxylic Acid, C11-15 Pareth-7 Carboxylic Acid, C12-13 Pareth-5 Carboxylic Acid, C12-13 Pareth-8 Carboxylic Acid, C12-13 Pareth-12 Carboxylic Acid, C12-15 Pareth-7 Carboxylic Acid, C12-15 Pareth-8 Carboxylic Acid, C14-15 Pareth-8 Carboxylic Acid, Deceth-7 Carboxylic Acid, Laureth-3 Carboxylic Acid, Laureth-4 Carboxylic Acid, Laureth-5 Carboxylic Acid, Laureth-6 Carboxylic Acid, Laureth-8 Carboxylic Acid, Laureth-10 Carboxylic Acid, Laureth-11 Carboxylic Acid, Laureth-12 Carboxylic Acid, Laureth-13 Carboxylic Acid, Laureth-14 Carboxylic Acid, Laureth-17 Carboxylic Acid, PPG-6-Laureth-6 Carboxylic Acid, PPG-8-Steareth-7 Carboxylic Acid, Myreth-3 Carboxylic Acid, Myreth-5 Carboxylic Acid, Nonoxynol-5 Carboxylic Acid, Nonoxynol-8 Carboxylic Acid, Nonoxynol-10 Carboxylic Acid, Octeth-3 Carboxylic Acid, Octoxynol-20 Carboxylic Acid, Oleth-3 Carboxylic Acid, Oleth-6 Carboxylic Acid, Oleth-10 Carboxylic Acid, PPG-3-Deceth-2 Carboxylic Acid, Capryleth-2 Carboxylic Acid, Ceteth-13 Carboxylic Acid, Deceth-2 Carboxylic Acid, Hexeth-4 Carboxylic Acid, Isosteareth-6 Carboxylic Acid, Isosteareth-11 Carboxylic Acid, Trudeceth-3 Carboxylic Acid, Trideceth-6 Carboxylic Acid, Trideceth-8 Carboxylic Acid, Trideceth-12 Carboxylic Acid, Trideceth-3 Carboxylic Acid, Trideceth-4 Carboxylic Acid, Trideceth-7 Carboxylic Acid, Trideceth-15 Carboxylic Acid, Trideceth-19 Carboxylic Acid, Undeceth-5 Carboxylic Acid, or mixtures thereof. In some cases, preferred ethoxylated acids include Oleth-10 Carboxylic Acid, Laureth-5 Carboxylic Acid, Laureth-11 Carboxylic Acid, or mixtures thereof.

### Acyl Amino Acids

Acyl amino acids that may be used include, but are not limited to, amino acid surfactants based on alanine, arginine, aspartic acid, glutamic acid, glycine, isoleucine, leucine, lysine, phenylalanine, serine, tyrosine, valine, sarcosine, threonine, and taurine. The most common cation associated with the acyl amino acid can be sodium or potassium. Alternatively, the cation can be an organic salt such as triethanolamine (TEA) or a metal salt.

Non-limiting examples of useful acyl amino acids include those of formula (VI):

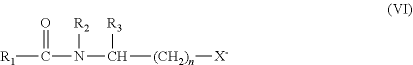

wherein:
R¹, R², and R³ are each independently selected from H or an alkyl chain having 1-24 carbon atoms, said chain being saturated or unsaturated, linear or branched, substituted or unsubstituted;
n ranges from 0 to 30; and
X is COO⁻ or SO₃⁻.

### Acyl Taurates

Non-limiting examples of acyl taurates include those of formula (VII):

RCO—NR¹CHR²CHR³SO³Na (VII)

wherein R, R¹, R², and R³ are each independently selected from H or an alkyl chain having from 1-24 carbon atoms, such as from 6-20 carbon atoms, or from 8-16 carbon atoms, said chain being saturated or unsaturated, linear or branched, substituted or unsubstituted.

In various embodiments, RCO— represents the coconut acid moiety. Non-limiting examples of acyl taurate salts include sodium cocoyl taurate and sodium methyl cocoyl taurate.

### Acyl Glycinates

Non-limiting examples of useful acyl glycinates include those of formula (VIII):

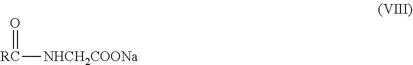

wherein R is an alkyl chain of 8 to 16 carbon atoms. Sodium is shown as the cation in the above formula (VIII), but the cation may be any alkali metal ion such as sodium or potassium, ammonium ions, or alkanolammonium ions such as monoethanolammonium or triethanolammonium ions.

Non-limiting examples of acyl glycinates include sodium cocoyl glycinate, sodium lauroyl glycinate, sodium myristoyl glycinate, potassium lauroyl glycinate, and potassium cocoyl glycinate.

### Acyl Glutamates

Non-limiting examples of useful acyl glutamates include those of formula (IX):

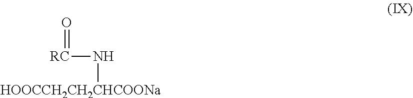

wherein R is an alkyl chain of 8 to 16 carbon atoms. Sodium is shown as the cation in the above formula (IX) but the cation may be any alkali metal ion such as sodium or potassium, ammonium ions, or alkanolammonium ions such as monoethanolammonium or triethanolammonium ions.

Non-limiting examples of acyl glutamates include dipotassium capryloyl glutamate, dipotassium undecylenoyl glutamate, disodium capryloyl glutamate, disodium cocoyl glutamate, disodium lauroyl glutamate, disodium stearoyl glutamate, disodium undecylenoyl glutamate, potassium capryloyl glutamate, potassium cocoyl glutamate, potassium lauroyl glutamate, potassium myristoyl glutamate, potassium stearoyl glutamate, potassium undecylenoyl glutamate, sodium capryloyl glutamate, sodium cocoyl glutamate, sodium lauroyl glutamate, sodium myristoyl glutamate, sodium olivoyl glutamate, sodium palmitoyl glutamate, sodium stearoyl glutamate, sodium undecylenoyl glutamate, triethanolamine mono-cocoyl glutamate, triethanolamine lauroylglutamate, and disodium cocoyl glutamate.

In various embodiments, the total amount of the at least one anionic surfactant in a composition disclosed herein may vary, but typically ranges from about 0.01% to about 6% by weight, including all subranges therebetween, such as from about 0.01% to about 5%, from 0.01% to about 4%, from about 0.01% to about 3%, from about 0.01% to about 2%, from about 0.01% to about 1.5%, from about 0.01% to about 1%, from about 0.1% to about 6%, from about 0.1% to about 5%, from 0.1% to about 4%, from about 0.1% to about 3%, from about 0.1% to about 2%, from about 0.1% to about 1.5%, from about 0.1% to about 1%, from about 0.5% to about 6%, from about 0.5% to about 5%, from about 0.5% to about 4%, from about 0.5% to about 3%, from about 0.5% to about 2%, from about 0.5% to about 1.5%, from about 0.5% to about 1%, or from about 0.75% to about 1% by weight, relative to the total weight of the composition. In some particular embodiments, the total amount of the at least one anionic surfactant in a composition disclosed herein may range from about 0.5% to about 1.5%, such as about 0.5% to about 1% by weight, relative to the total weight of the composition.

### Silicones

Compositions according to the disclosure comprise at least one silicone compound. Without intending to be limited by theory, the at least one silicone may provide benefits such as flexibility, manageability, discipline, an overall softer end feel, etc. In various embodiments, the at least one silicone may be chosen from dimethicone, dimethicone copolyols, dimethicone copolymers, amino functional silicones, and mixtures thereof.

In some embodiments, the at least one silicone compound is chosen from dimethicone copolyols. Dimethicone copolyol is a general term used for a group of polymers made from dimethicone and polyoxyethylene and/or polyoxypropylene. Suitable examples of dimethicone copolyols include Dimethicone PEG-8 Adipate, Dimethicone PEG-8 Benzoate, Dimethicone PEG-7 Phosphate, Dimethicone PEG-8 Phosphate, Dimethicone PEG-10 Phosphate, Dimethicone PEG/PPG-20/23 Benzoate, Dimethicone PEG/PPG-7/4 Phosphate, Dimethicone PEG/PPG-12/4 Phosphate, PEG-3 Dimethicone, PEG-7 Dimethicone, PEG-8 Dimethicone, PEG-9 Dimethicone, PEG-10 Dimethicone, PEG-12 Dimethicone, PEG-14 Dimethicone, PEG-17 Dimethicone, PEG/PPG-3/10 Dimethicone, PEG/PPG-4/12 Dimethicone, PEG/PPG-6/11 Dimethicone, PEG/PPG-8/14 Dimethicone, PEG/PPG-14/4 Dimethicone, PEG/PPG-15/15 Dimethicone, PEG/PPG-16/2 Dimethicone, PEG/PPG-17/18 Dimethicone, PEG/PPG-18/18 Dimethicone, PEG/PPG-19/19 Dimethicone, PEG/PPG-20/6 Dimethicone, PEG/PPG-20/15 Dimethicone, PEG/PPG-20/20 Dimethicone, PEG/PPG-20/23 Dimethicone, PEG/PPG-20/29 Dimethicone, PEG/PPG-22/23 Dimethicone, PEG/PPG-22/24 Dimethicone, PEG/PPG-23/6 Dimethicone, PEG/PPG-25/25 Dimethicone, PEG/PPG-27/27 Dimethicone, and mixtures thereof

In an embodiment, the at least one silicone compound may be an amino functional silicone. In an embodiment, the at least one silicone compound of the present disclosure is amino functional silicone comprising at least one functionalized amodimethicone. The term “amino functional silicone” as used herein can mean any silicone comprising at least one primary, secondary or tertiary amine or a quaternary ammonium group (i.e., a quaternized group).

Non-limiting examples of amino functional silicone that may be used include:

a) polysiloxanes corresponding to formula (A):

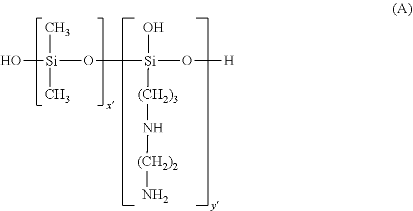

wherein x′ and y′ are integers such that the weight-average molecular weight (Mw) is comprised between about 5000 and 500 000;

b) amino silicones corresponding to formula (B):

R′ₐG₃₋ₐ—Si(OSiG₂)ₙ—(OSiG_{b}R′_{2-b})ₘ—O—SiG₃₋ₐ−R′ₐ (B)

wherein:

G, which may be identical or different, designate a hydrogen atom, or a phenyl, OH or C₁-C₈ alkyl group, for example methyl, or C₁-C₈ alkoxy, for example methoxy,

a, which may be identical or different, denote the number 0 or an integer from 1 to 3, in particular 0;

b denotes 0 or 1, and in particular 1;

m and n are numbers such that the sum (n+m) ranges from 1 to 2000 and in particular from 50 to 150, it being possible for n to denote a number from 0 to 1999 and in particular from 49 to 149, and form to denote a number from 1 to 2000 and in particular from 1 to 10; and

R′, which may be identical or different, denote a monovalent radical having formula —CqH₂qL, in which q is a number ranging from 2 to 8 and L is an optionally quaternized amino group chosen from the following groups:
—NR″—Q—N(R″)₂
—N(R″)₂
—N+(R″)₃A-
—N+H(R″)₂A-
—N+H₂(R″) A-
—N(R″)—Q-N+R″H₂A-
—NR″-Q-N+(R″)₂H A-
—NR″-Q-N+(R″)₃A-,
in which R″, which may be identical or different, denote hydrogen, phenyl, benzyl, or a saturated monovalent hydrocarbon-based radical, for example a C₁-C₂₀ alkyl radical; Q denotes a linear or branched CrH₂ᵣ group, r being an integer ranging from 2 to 6, preferably from 2 to 4; and A-represents a cosmetically acceptable ion, in particular a halide such as fluoride, chloride, bromide or iodide.

A group of amino silicones corresponding to formula (B) is represented by the silicones called “trimethylsilylamodimethicone” having formula (C):

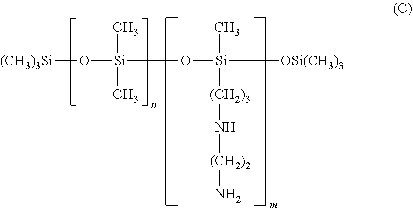

in which n and m have the meanings as in formula B.

Another group of amino silicones corresponding to formula (B) is represented by silicones of formula (D):

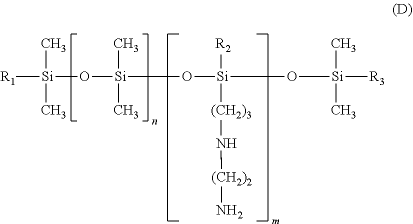

wherein:

m and n are numbers such that the sum (n+m) can range from 1 to 1000, in particular from 50 to 250 and more particularly from 100 to 200, it being possible for n to denote a number from 0 to 999 and in particular from 49 to 249, and more particularly from 125 to 175, and for m to denote a number from 1 to 1000 and in particular from 1 to 10, and more particularly from 1 to 5; and

R1, R2, R3, which may be identical or different, represent a hydroxy or C1-C4 alkoxy radical, where at least one of the radicals R1 to R3 denotes an alkoxy radical.

In one embodiment, the alkoxy radical is preferably a methoxy radical. In further embodiments, the hydroxy/alkoxy mole ratio ranges preferably from 0.2:1 to 0.4:1 and preferably from 0.25:1 to 0.35:1 and more particularly equals 0.3:1. In various embodiments, the weight-average molecular weight (Mw) of the silicone ranges from 2000 to 1,000,000, such as from 3500 to 200,000.

Another group of amino silicones corresponding to formula (B) is represented by silicones of formula (E):

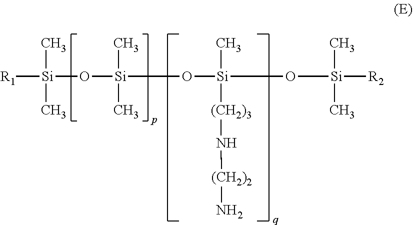

wherein:

p and q are numbers such that the sum (p+q) ranges from 1 to 1000, particularly from 50 to 350, and more particularly from 150 to 250; it being possible for p to denote a number from 0 to 999 and in particular from 49 to 349, and more particularly from 159 to 239 and for q to denote a number from 1 to 1000, in particular from 1 to 10, and more particularly from 1 to 5; and

R₁, R₂, which are different, represent a hydroxy or C₁-C₄ alkoxy radical, where at least one of the radicals R₁ or R₂ denotes an alkoxy radical.

In one embodiment, the alkoxy radical is preferably a methoxy radical. In further embodiments, the hydroxy/alkoxy mole ratio ranges generally from 1:0.8 to 1:1.1 and preferably from 1:0.9 to 1:1 and more particularly equals 1:0.95. In various embodiments, the weight-average molecular weight (Mw) of the silicone ranges preferably from 2000 to 200,000, even more particularly 5000 to 100,000 and more particularly from 10,000 to 50,000.

Commercial products corresponding to these silicones having structure (D) or (E) may include in their composition one or more other amino silicones whose structure is different than formulae (D) or (E). For example, a product containing amino silicones having structure (D) is sold by Wacker under the name Belsil® ADM 652, and products containing amino silicones having structure (E) include those sold by Wacker under the names Fluid WR 1300® or Finish CT 96 E® or SLM 28020®.

Another group of amino silicones corresponding to formula (B) is represented by the following formula (F):

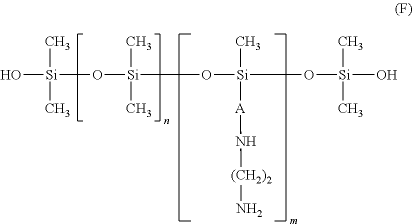

wherein:

m and n are numbers such that the sum (n+m) ranges from 1 to 2000 and in particular from 50 to 150, it being possible for n to denote a number from 0 to 1999 and in particular from 49 to 149, and form to denote a number from 1 to 2000 and in particular from 1 to 10; and

A denotes a linear or branched alkylene radical containing from 4 to 8 carbon atoms and preferably 4 carbon atoms. This radical is preferably linear.

The weight-average molecular weight (Mw) of these amino silicones ranges preferably from 2000 to 1,000,000 and even more particularly from 3500 to 200,000. A preferred silicone of formula (F) is amodimethicone (INCI name) sold under the tradename XIAMETER® MEM-8299 Cationic Emulsion by Dow Corning or sold under the tradename SILSOFT 253, by Momentive Performance Materials.

Another group of amino silicones corresponding to formula (B) is represented by the following formula (G):

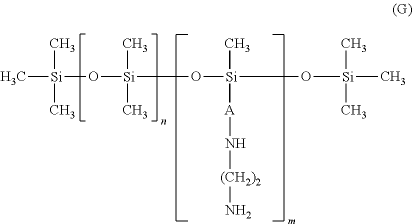

wherein:

m and n are numbers such that the sum (n+m) ranges from 1 to 2000 and in particular from 50 to 150, it being possible for n to denote a number from 0 to 1999 and in particular from 49 to 149, and form to denote a number from 1 to 2000 and in particular from 1 to 10; and

A denotes a linear or branched alkylene radical containing from 4 to 8 carbon atoms and preferably 4 carbon atoms. This radical is preferably branched.

The weight-average molecular weight (Mw) of these amino silicones ranges preferably from 500 to 1,000,000 and even more particularly from 1000 to 200,000. Commercially available silicones having this formula include DC2-8566 Amino Fluid by Dow Corning.

c) amino silicones corresponding to formula (H):

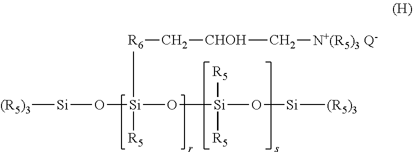

wherein:

R5 represents a monovalent hydrocarbon-based radical containing from 1 to 18 carbon atoms, and in particular a C₁-C₁₈ alkyl or C₂-C₁₈ alkenyl radical, for example methyl;

R6 represents a divalent hydrocarbon-based radical, in particular a C₁-C₁₈ alkylene radical or a divalent C₁-C₁₈, for example C₁-C₈, alkylenoxy radical linked to the Si via an SiC bond;

Q⁻ is an anion such as a halide ion, in particular chloride, or an organic acid salt (for example acetate);

r represents a mean statistical value from 2 to 20 and in particular from 2 to 8; and

s represents a mean statistical value from 20 to 200 and in particular from 20 to 50.

Examples of such amino silicones are described in U.S. Pat. No. 4,185,087.

d) quaternary ammonium silicones having formula (I):

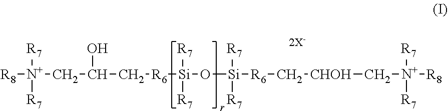

wherein:

R₇, which may be identical or different, represent a monovalent hydrocarbon-based radical containing from 1 to 18 carbon atoms, and in particular a C₁-C₁₈ alkyl radical, a C₂-C₁₈ alkenyl radical or a ring containing 5 or 6 carbon atoms, for example methyl;

R6 represents a divalent hydrocarbon-based radical, in particular a C₁-C₁₈ alkylene radical or a divalent C₁-C₁₈, for example C₁-C₈, alkylenoxy radical linked to the Si via an SiC bond;

R₈, which may be identical or different, represent a hydrogen atom, a monovalent hydrocarbon-based radical containing from 1 to 18 carbon atoms, and in particular a C₁-C₁₈ alkyl radical, a C₂-C₁₈ alkenyl radical or a —R₆—NHCOR₇ radical;

X⁻ is an anion such as a halide ion, in particular chloride, or an organic acid salt (for example acetate); and

r represents a mean statistical value from 2 to 200 and in particular from 5 to 100.

Examples of such silicones are described, for example, in EP-A 0 530 974.

e) amino silicones having formula (J):

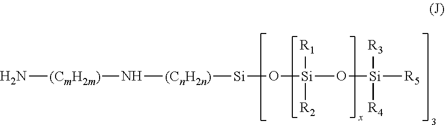

wherein:

R₁, R₂, R₃ and R₄, which may be identical or different, denote a C₁-C₄ alkyl radical or a phenyl group;

R₅ denotes a C₁-C₄ alkyl radical or a hydroxyl group;

n is an integer ranging from 1 to 5;

m is an integer ranging from 1 to 5; and

x is chosen such that the amine number is between 0.01 and 1 meq/g.

f) multiblock polyoxyalkylenated amino silicones, of type (AB)ₙ, A being a polysiloxane block and B being a polyoxyalkylenated block containing at least one amine group. In various embodiments, such silicones may comprise repeating units having one of the following general formulae:

[—(SiMe₂O)xSiMe₂—R—N(R″)—R′—O(C₂H₄₀)ₐ(C₃H₆O)_{b}—R′—N(H)—R—]

[—(SiMe₂O)xSiMe₂—R—N(R″)—R′—O(C₂H₄O)ₐ(C₃H₆O)b—]

wherein:

a is an integer greater than or equal to 1, preferably ranging from 5 to 200, more particularly ranging from 10 to 100;

b is an integer comprised between 0 and 200, preferably ranging from 4 to 100, more particularly between from 5 and 30;

x is an integer ranging from 1 to 10,000, more particularly from 10 to 5000;

R″ is a hydrogen atom or a methyl;

R, which may be identical or different, represent a divalent linear or branched C₂-C₁₂ hydrocarbon-based radical, optionally including one or more heteroatoms such as oxygen; preferably, R denotes an ethylene radical, a linear or branched propylene radical, a linear or branched butylene radical, or a —CH₂CH₂CH₂OCH(OH)CH₂— radical; preferentially R denotes a —CH₂CH₂CH₂OCH(OH)CH₂— radical; and

R′, which may be identical or different, represent a divalent linear or branched C₂-C₁₂ hydrocarbon-based radical, optionally including one or more heteroatoms such as oxygen; preferably, R′ denotes an ethylene radical, a linear or branched propylene radical, a linear or branched butylene radical, or a —CH₂CH₂CH₂OCH(OH)CH₂— radical; preferentially R′ denotes —CH(CH₃)—CH₂—.

The siloxane blocks preferably represent from 50-95 mol % of the total weight of the silicone, more particularly from 70-85 mol %. The amine content is preferably from 0.02 to 0.5 meq/g of copolymer in a 30% solution in dipropylene glycol, more particularly from 0.05 to 0.2. The weight-average molecular weight (Mw) of the silicone is preferably comprised between 5000 and 1,000,000, more particularly between 10,000 and 200,000. Non-limiting examples include bis-amino PEG/PPG-41/3 aminoethyl PG-propyl dimethicone and PEG-40/PPG-8 methylaminopropyl hydroxypropyl dimethicone copolymer. Commercially available products include the silicones sold under the names SILSOFT A-843 or SILSOFT A+ by Momentive.

g) the alkylamino silicones corresponding to formula (K) below:

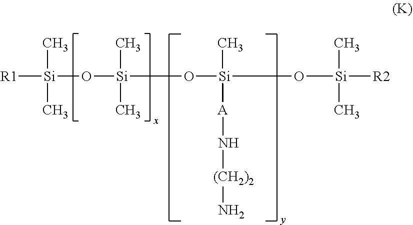

wherein:

x and y are numbers ranging from 1 to 5000; preferably, x ranges from 10 to 2000 and especially from 100 to 1000; preferably, y ranges from 1 to 100;

R₁ and R₂, which may be identical or different, preferably identical, are linear or branched, saturated or unsaturated alkyl radicals, comprising 6 to 30 carbon atoms, preferably 8 to 24 carbon atoms and especially 12 to 20 carbon atoms; and

A denotes a linear or branched alkylene radical containing from 2 to 8 carbon atoms.

In various exemplary embodiments, A comprises from 3 to 6 carbon atoms, especially 4 carbon atoms, and in certain embodiments, A is branched. Mention may be made of the following divalent radicals: —CH₂CH₂CH₂ and —CH₂CH(CH₃)CH₂—.

Preferably, R₁ and R₂, which may be identical or different, are saturated linear alkyl radicals comprising 6 to 30 carbon atoms, preferably 8 to 24 carbon atoms and especially 12 to 20 carbon atoms; mention may be made in particular of dodecyl, tetradecyl, pentadecyl, hexadecyl, heptadecyl, octadecyl, nonadecyl and eicosyl radicals; and preferentially, R₁ and R₂, which may be identical or different, are chosen from hexadecyl (cetyl) and octadecyl (stearyl) radicals.

In various exemplary embodiments, the silicone of formula (K) is chosen such that:

x ranges from 10 to 2000 and especially from 100 to 1000;

y ranges from 1 to 100;

A comprises 3 to 6 carbon atoms and especially 4 carbon atoms; preferably, A is branched; and more particularly A is chosen from the following divalent radicals: CH₂CH₂CH₂ and —CH₂CH(CH₃)CH₂—; and

R₁ and R₂, which may be identical or different, are linear, saturated alkyl radicals comprising 6 to 30 carbon atoms, preferably 8 to 24 carbon atoms and especially 12 to 20 carbon atoms; chosen in particular from dodecyl, tetradecyl, pentadecyl, hexadecyl, heptadecyl, octadecyl, nonadecyl and eicosyl radicals; preferentially, R₁ and R₂, which may be identical or different, being chosen from hexadecyl (cetyl) and octadecyl (stearyl) radicals.

An exemplary amino silicone of formula (K) is bis-cetearylamodimethicone (INCI name), such as the silicone sold under the name SILSOFT AX by Momentive.

h) silicone compounds with at least one quaternary ammonium group. Suitable non-limiting examples are quaternium-80, silicone quaternium-1, silicone quaternium-2, silicone quaternium-2 panthenol succinate, silicone quaternium-3, silicone quaternium-4, silicone quaternium-5, silicone quaternium-6, silicone quaternium-7, silicone quaternium-8, silicone quaternium-9, silicone quaternium-10, silicone quaternium-11, silicone quaternium-12, silicone quaternium-15, silicone quaternium-16, silicone quaternium-16/Glycidoxy Dimethicone Crosspolymer, silicone quaternium-17, silicone quaternium-18, silicone quaternium-20 and silicone quaternium-21. Preferred are quaternium 80, silicone quaternium-16, silicone quaternium-18, silicone quaternium-1, silicone quaternium-2, silicone quaternium-3, silicone quaternium-4, silicone quaternium-5, silicone quaternium-6, silicone quaternium-7, silicone quaternium-8, silicone quaternium-9, silicone quaternium-10, silicone quaternium-11, silicone quaternium-12, silicone quaternium-15, silicone quaternium-17, silicone quaternium-20 and silicone quaternium-21. More preferred are quaternium-80, silicone quaternium-16, silicone quaternium-18, silicone quaternium-3, silicone quaternium-4, silicone quaternium-5, silicone quaternium-6, silicone quaternium-7, silicone quaternium-8, silicone quaternium-9, silicone quaternium-10, silicone quaternium-11, silicone quaternium-12, silicone quaternium-15, and silicone quaternium-17. For example, quaternium-80, silicone quaternium-16, silicone quaternium-18, silicone quaternium-15, or mixtures thereof may be chosen.

The amount of the at least one silicone copolymer that may be included in various embodiments can vary, but typically ranges from about 0.01% to about 10%, based on the total weight of the composition, including all ranges and subranges therebetween. For example, the total amount of the at least silicone may range from about 0.01% to about 8%, from about 0.01% to about 5%, from about 0.01% to about 4%, from about 0.01% to about 3%, from about 0.01% to about 2%, from about 0.1% to about 10%, from about 0.1% to about 8%, from about 0.1% to about 5%, from about 0.1% to about 4%, from about 0.1% to about 3%, from about 0.1% to about 2%, from about 0.5% to about 10%, from about 0.5% to about 8%, from about 0.5% to about 5%, from about 0.5% to about 4%, from about 0.5% to about 3%, from about 0.5% to about 2%, from about 1 to about 10%, from about 1% to about 8%, from about 1% to about 5%, from about 1% to about 4%, from about 1% to about 3%, or from about 1% to about 2%, by weight, relative to the total weight of the composition. In some particular embodiments, the total amount of the at least one silicone may range from about 1% to about 3%, or about 1.5% to about 2.5% by weight, relative to the total weight of the composition.

### Polyols

Compositions according to the disclosure comprise at least one polyol. In certain embodiments, compositions comprise at least two polyols. The term “polyol,” as used herein, refers to an organic molecule comprising at least two free hydroxy groups, such as, for example, at least three free hydoxy groups. The polyols may be liquid at ambient temperature (25° C.). The polyols may have from 2 to 32 carbon atoms, such as from 3 to 16 carbon atoms, or from 3 to 12 carbon atoms.

By way of example, the at least one polyol may be chosen from sugar alcohols or alkane polyols. Mixtures of polyols, such as mixtures of sugar alcohols and/or alkane polyols, may also be used. In certain embodiments, compositions comprise at least two polyols wherein at least one polyol is chosen from sugar alcohols, and at least one polyol is chosen from alkane polyols.

Sugar alcohols that can be used include but are not limited to sorbitol, maltitol, maltotriitol, erythritol, arabitol, adonitol, dulcitol, glucose, fructose, xylose, trehalose, sucrose, maltose, saccharose, lactose, and other hydrogenated oligosaccharides and polysaccharides, and mixtures thereof.

Exemplary and non-limiting alkane polyols that can be used in the compositions according to the present disclosure include glycerin, 1,2,6-hexanetriol, trimethylolpropane, ethylene glycol, propylene glycol, diethylene glycol, triethylene glycol, tetraethylene glycol, pentaethylene glycol, dipropylene glycol, 2-butene-1,4-diol, 2-ethyl-1,3-hexanediol, 2-methyl-2,4-pentanediol, caprylyl glycol, 1,2-hexanediol, 1,2-pentanediol, and 4-methyl-1,2-pentanediol, or a mixture thereof.

The amount of the at least one polyol may vary but typically ranges from about 0.01% to about 15%, including all subranges therebetween, such as from about 0.1% to about 15%, from about 0.1% to about 12%, from about 0.1% to about 10%, from about 0.1% to about 8%, from about 0.1% to about 5%, from about 0.1% to about 4%, from about 0.1% to about 3%, from about 0.1% to about 2%, from about 0.1% to about 1.5%, from about 0.1% to about 1%, from about 0.5% to about 15%, from about 0.5% to about 12%, from about 0.5% to about 10%, from about 0.5% to about 8%, from about 0.5% to about 5%, from about 0.5% to about 4%, from about 0.5% to about 3%, from about 0.5% to about 2%, from about 0.5% to about 1.5%, from about 0.5% to about 1%, from about 1% to about 15%, from about 1% to about 12%, from about 1% to about 10%, from about 1% to about 9%, from about 1% to about 8%, from about 1% to about 7%, from about 1% to about 6%, from about 1% to about 5%, from about 1% to about 4%, from about 1% to about 3%, from about 1% to about 2%, from about 1.5% to about 15%, from about 1.5% to about 12%, from about 1.5% to about 10%, from about 1.5% to about 9%, from about 1.5% to about 8%, from about 1.5% to about 7%, from about 1.5% to about 6%, from about 1.5% to about 5%, from about 1.5% to about 4%, from about 1.5% to about 3%, or from about 1.5% to about 2% by weight, relative to the total weight of the composition. For example, the at least one polyol may be present in an amount ranging from about 1 to about 9%, such as about 1.5% to about 8.5% by weight, relative to the total weight of the composition.

In certain exemplary embodiments, the at least one polyol is a sugar alcohol and is present in an amount ranging from about 0.05% to about 5%, including all subranges therebetween, such as from about 0.1% to about 5%, from about 0.1% to about 4%, from about 0.1% to about 3%, from about 0.1% to about 2.5%, from about 0.1% to about 2%, from about 0.1% to about 1.5%, from about 0.1% to about 1%, from about 0.5% to about 5%, from about 0.5% to about 4%, from about 0.5% to about 3%, from about 0.5% to about 2.5%, from about 0.5% to about 2%, from about 0.5% to about 1.5%, or from about 0.5% to about 1%, by weight, relative to the total weight of the composition.

In some particular embodiments, compositions according to the disclosure comprise sugar alcohol(s) present in the composition in an amount ranging from about 0.5% to about 1.5% by weight, relative to the total weight of the composition, and the sugar alcohol(s) comprises sorbitol.

In certain further exemplary embodiments, the at least one polyol is an alkane polyol, and is present in the composition in an amount ranging from about 0.1% to about 15%, such as from about 0.5% to about 10%, from about 0.5% to about 9%, from about 0.5% to about 8%, from about 0.5% to about 7%, from about 0.5% to about 6%, from about 0.5% to about 5%, from about 0.5% to about 4%, from about 0.5% to about 3%, from about 0.5% to about 2%, from about 1% to about 10%, from about 1% to about 9%, from about 1% to about 8%, from about 1% to about 7%, from about 1% to about 6%, from about 1% to about 5%, from about 1% to about 4%, from about 1% to about 3%, or from about 1% to about 2% by weight, relative to the total weight of the composition.

In some particular embodiments, compositions according to the disclosure comprise alkane polyol(s) present in the composition in an amount ranging from about 1% to about 7% by weight, relative to the total weight of the composition, and the alkane polyol(s) comprises glycerin.

### Additional Components

Compositions according to the disclosure may comprise additional components, including but not limited to solvents, conditioning agents, active agents, preservatives, and auxiliary components.

### Solvents

Compositions according to the disclosure comprise a solvent. The solvent may be chosen from water, non-aqueous solvents, or mixtures thereof.

In some embodiments, the solvent comprises, consists essentially of, or consists of water. The total amount of water in the compositions may vary depending on the type of composition and the desired consistency, viscosity, etc.

In certain embodiments, the composition comprises one or more non-aqueous solvents, other than or in addition to polyols discussed above. For example, glycerin, C₁₋₄ alcohols, organic solvents, fatty alcohols, fatty ethers, fatty esters, polyols, glycols, vegetable oils, mineral oils, liposomes, laminar lipid materials, or any a mixture thereof. Non-limiting examples of solvents which may be used include alkane polyols such as glycerin, 1,2,6-hexanetriol, trimethylolpropane, ethylene glycol, propylene glycol, diethylene glycol, triethylene glycol, tetraethylene glycol, pentaethylene glycol, dipropylene glycol, 2-butene-1,4-diol, 2-ethyl-1,3-hexanediol, 2-methyl-2,4-pentanediol, caprylyl glycol, 1,2-hexanediol, 1,2-pentanediol, and 4-methyl-1,2-pentanediol; alkyl alcohols having 1 to 4 carbon atoms such as ethanol, methanol, butanol, propanol, and isopropanol; glycol ethers such as ethylene glycol monomethyl ether, ethylene glycol monoethyl ether, ethylene glycol monobutyl ether, ethylene glycol monomethyl ether acetate, diethylene glycol monomethyl ether, diethylene glycol monoethyl ether, diethylene glycol mono-n-propyl ether, ethylene glycol mono-iso-propyl ether, diethylene glycol mono-iso-propyl ether, ethylene glycol mono-n-butyl ether, ethylene glycol mono-t-butyl ether, diethylene glycol mono-t-butyl ether, 1-methyl-1-methoxybutanol, propylene glycol monomethyl ether, propylene glycol monoethyl ether, propylene glycol mono-t-butyl ether, propylene glycol mono-n-propyl ether, propylene glycol mono-iso-propyl ether, dipropylene glycol monomethyl ether, dipropylene glycol monoethyl ether, dipropylene glycol mono-n-propyl ether, and dipropylene glycol mono-iso-propyl ether; 2-pyrrolidone, N-methyl-2-pyrrolidone, 1,3-dimethyl-2-imidazolidinone, formamide, acetamide, dimethyl sulfoxide, sorbit, sorbitan, acetine, diacetine, triacetine, sulfolane, and a mixture thereof.

The solvent may be present in the composition in an amount ranging from about 60% to about 98% by weight, relative to the total weight of the composition, including all ranges and subranges therebetween. For example, in one embodiment, the total amount of solvent may be about 65% to about 95%, about 65% to about 90%, about 70% to about 95%, about 70% to about 90%, about 75% to 95%, or about 75% to 90% by weight, relative to the total weight of the composition. In certain embodiments, the solvent is primarily comprised of water, such as from about 90% to about 99%, or about 95% to about 99%, of the total solvent.

### Conditioning Agents

Compositions according to the disclosure may optionally comprise at least one conditioning agent other than silicone copolymers described above.

In various exemplary embodiments, the at least one conditioning agent may be chosen from non-silicone fatty compounds. The term “non-silicone fatty compound” means a fatty compound that does not contain any silicon atoms (Si). Non-limiting examples of non-silicone fatty compounds include oils, mineral oil, fatty alcohols, fatty acids, fatty alcohol derivatives, fatty acid derivatives (such as alkoxylated fatty acids or polyethylene glycol esters of fatty acids, propylene glycol esters of fatty acids, butylene glycol esters of fatty acids, esters of neopentyl glycol and fatty acids, polyglycerol/glycerol esters of fatty acids, glycol diesters or diesters of ethylene glycol, fatty acids or esters of fatty acids and fatty alcohols, esters of short chain alcohols and fatty acids), esters of fatty alcohols, hydroxy-substituted fatty acids, waxes, triglyceride compounds, lanolin, and a mixture thereof. Non-limiting examples of the fatty alcohols, fatty acids, fatty alcohol derivatives, and fatty acid derivatives are found in International Cosmetic Ingredient Dictionary, Sixteenth Edition, 2016, which is incorporated by reference herein in its entirety.

Mineral oils, such as liquid paraffin or liquid petroleum, or animal oils, such as perhydrosqualene or arara oil, or alternatively of vegetable oils, such as sweet almond, calophyllum, palm, castor, avocado, jojoba, olive or cereal germ oil, may be utilized. It is also possible to use esters of these oils, e.g., jojoba esters. Also useful are esters of lanolic acid, of oleic acid, of lauric acid, of stearic acid or of myristic acid; esters of alcohols, such as oleyl alcohol, linoleyl or linolenyl alcohol, isostearyl alcohol or octyldodecanol; and/or acetylglycerides, octanoates, decanoates or ricinoleates of alcohols or of polyalcohols.

In various exemplary embodiments, the conditioning agents may be chosen from cationic polymers, although in certain embodiments the compositions are free or essentially free of cationic polymers. The term “cationic polymer” means any polymer comprising at least one cationic group and/or at least one group that may be ionized into a cationic group.

In various embodiments, the total amount of at least one conditioning agent in the compositions may vary, but is typically from about 0.01% to about 10% by weight, including all subranges therebetween, such as from about 0.01% to about 8%, from about 0.01% to about 5%, from about 0.01% to about 3%, from about 0.01% to about 1%, from about 0.5% to about 10%, from about 0.5% to about 8%, from about 0.5% to about 5%, from about 1% to about 10%, from about 1% to about 8%, from about 1% to about 5% by weight, relative to the total weight of the composition. In some embodiments, the total amount of conditioning agents are present in an amount ranging from about 0.05% to about 5% by weight, based on the total weight of the composition.

### Active Agents

In various embodiments, compositions according to the disclosure may optionally comprise at least one active agent such as an acid or sodium hydroxide, or mixture thereof, to provide optimized strengthening benefits to the hair. Non-limiting examples of useful acids include glycolic acid, lactic acid, malic acid, tartaric acid, citric acid, ascorbic acid, mandelic acid, azelaic acid, glyceric acid, tartronic acid, gluconic acid, benzylic acid, pyruvic acid, 2-hydroxybutyric acid, salicylic acid, trichloroacetic acid, or mixtures thereof.

The acids are typically non-polymeric and may have one (mono), two (di), or three (tri) carboxylic acid groups (—COOH). The non-polymeric mono-, di-, and tricarboxylic acids, and/or salts thereof, typically have a molecular weight of less than about 500 g/mol, less than about 400 g/mol, or less than about 300 g/mol.

Non-limiting examples of monocarboxylic acids include formic acid, acetic acid, propionic acid, butyric acid, valeric acid, caproic acid, entanthic acid, caprylic acid, pelargonic acid, capric acid, undecylic acid, lauric acid, tridecylic acid, lauric acid, tridecylic acid, myristic acid, pentadecylic acid, palmitic acid, margaric acid, stearic acid, nonadecylic acid, arachidic acid, lactic acid, a salt thereof, and a mixture thereof.

Non-limiting examples of dicarboxylic acids include oxalic acid, malonic acid, malic acid, glutaric acid, citraconic acid, succinic acid, adipic acid, tartaric acid, fumaric acid, maleic acid, sebacic acid, azelaic acid, dodecanedioic acid, phthalic acid, isophthalic acid, terephthalic acid, 2,6-naphthalene dicarboxylic acid, a salt thereof, and a mixture thereof.

Non-limiting examples of tricarboxylic acids include citric acid, isocitric acid, aconitric acid, propane-1,2,3-tricarboxylic acid, benzene-1,3,5-tricarboxylic acid, a salt thereof, and a mixture thereof.

If present, the total amount of the one or more active agents may vary but typically ranges from about 0.0001% to about 10%, such as from about 0.0001% to about 5%, about 0.0001% to about 1%, about 0.001% to about 10%, about 0.001% to about 5%, about 0.001% to about 1% by weight, about 0.01% to about 10%, about 0.01% to about 5%, about 0.01% to about 1%, about 0.1% to about 10%, about 0.1% to about 5%, or about 0.1% to about 1% by weight, based on the total weight of the composition. For example, the total amount of the one or more acids may range from about 0.0001% to about 0.5% by weight, based on the total weight of the composition.

### Preservatives

One or more preservatives may be included in the compositions described herein for treating hair. Suitable preservatives include, but are not limited to, glycerin containing compounds (e.g., glycerin or ethylhexylglycerin or phenoxyethanol), benzyl alcohol, parabens (methylparaben, ethylparaben, propylparaben, butylparaben, isobutylparaben, etc.), sodium benzoate, benzoic acid, chlorhexidine digluconate, ethylenediamine-tetraacetic acid (EDTA), potassium sorbate, and/or grapefruit seed extract, or a mixture thereof. Other preservatives are known in the cosmetics industries and include salicylic acid, DMDM Hydantoin, Formaldahyde, Chlorphenism, Triclosan, Imidazolidinyl Urea, Diazolidinyl Urea, Sorbic Acid, Methylisothiazolinone, Sodium Dehydroacetate, Dehydroacetic Acid, Quaternium-15, Stearalkonium Chloride, Zinc Pyrithione, Sodium Metabisulfite, 2-Bromo-2-Nitropropane, Chlorhexidine Digluconate, Polyaminopropyl biguanide, Benzalkonium Chloride, Sodium Sulfite, Sodium Salicylate, Citric Acid, Neem Oil, Essential Oils (various), Lactic Acid, Vitamin E (tocopherol), and a mixture thereof. In some cases, the hair-treatment compositions may include one or more preservatives selected from the group consisting of sodium benzoate, benzoic acid, chlorhexidine digluconate, chlorhexidine dihydrochloride, salicylic acid, phenoxyethanol, methyl paraben, and a mixture thereof.

The total amount of the one or more preservatives, when present, may vary. In some cases, the total amount of the one or more preservatives is about 0.01% to about 5%, about 0.01% to about 4%, about 0.15% to about 1%, or about 1% to about 3%, by weight, relative to the total weight of the composition.

### Auxiliary Components

Compositions according to the disclosure may optionally comprise any auxiliary component suitable for use in such compositions. Such components may include, but are not limited to, dyes/pigments, moisturizing agents, fatty substances, thickeners other than those previously described, fillers, structuring agents, shine agents, antioxidants or reducing agents, penetrants, sequestrants, fragrances, buffers, dispersants, plant extracts, preserving agents, opacifiers, sunscreen agents, vitamins, pH adjusting agents, and antistatic agents.

Optional auxiliary components may be present in an amount ranging up to about 15% by weight, relative to the total weight of the composition.

Optionally, the compositions may comprise up to about 100% biodegradable, sustainable, and/or environmentally friendly raw materials, such as up to about 99%, up to about 98%, up to about 97%, up to about 96%, or up to about 95%.

In various embodiments, the compositions may have a pH less than or equal to about 7. For example, the pH of the composition may range from about 2 to about 6, such as from about 3 to about 5, or from about 3.5 to about 4.5, including all ranges and subranges therebetween.

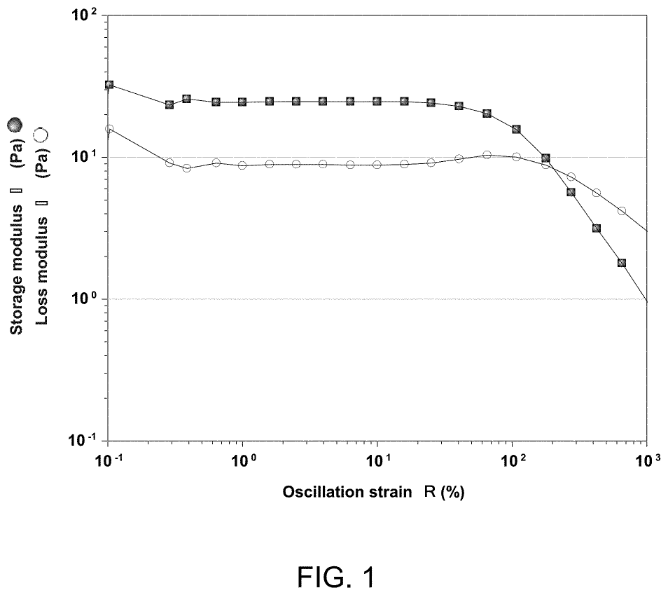

Compositions according to the disclosure are typically in the form of a gel prior to application of shear stress. The term “gel” is generally understood to mean a soft, semi-solid, or solid-like material that exhibits low to no flow under steady state. A gel can also be understood based on dynamic strain sweep test using a rheometer. FIG. 1 illustrates the typical rheological property of a general gel at the temperature where composition is applied. As shown in FIG. 1, the application of oscillating strains to a material allows to obtain two dynamic moduli, namely, the storage modulus, G′, a measure of the elasticity, and the loss modulus, G″, representing viscous components, at a given frequency, w, of oscillation. A gel will typically exhibit a semi-solid-like or solid-like mechanical spectrum, that is, G′>G″ throughout the linear viscoelastic region.

In various embodiments, the gel composition may have the appearance of a traditional gel, and be semi-clear or substantially clear. The viscosity of the gel is not limited, but by way of example only, in certain embodiments the viscosity may range from about 1 to about 25 Pa*s, such as about 3.5 to about 23 Pa*s, about 5 to about 15 Pa*s, about 8 to about 10 Pa*s, or about 9 Pa*s, including all ranges and subranges therebetween, at a shear rate 2 1/s when measured using a rheometer at 25° C. with a 40 mm parallel plate and 1000 μm gap. By way of non-limiting example only, in one embodiment, the viscosity of the gel may range up to about 10 Pa*s, and upon application of shear stress, the viscosity of the composition decreases to less than 0.1 Pa*s and a foam is formed.

In at least certain embodiments, the compositions in gel form may be stable, meaning that no or essentially no phase separation or significant change in pH or viscosity is seen, over a period of at least one week, such as at least one month, at least 6 months, or at least one year under ambient conditions.

Compositions in the form of a gel according to the disclosure are able to transform into a foam (they have decreased viscosity) upon application of sufficient shear stress. For example, mechanical shear stress, such as that which is applied by a pump dispenser, may effect the transformation of the gel to a foam. Other means of applying shear stress, for example shaking the composition, may also cause the gel to transform into a foam.

One way in which foam density is described herein relates to the volume of air bubbles to the total volume of the foam, which includes the bubbles and the liquid carrier thereof. The foam of compositions according to the disclosure may thus comprise air bubbles in at least about 30%, such as at least about 40%, of the foam, and the density may thus be described as at least about 30% or at least about 40% v/v. In various embodiments, the foams may be creamy and/or fine-pored. In certain embodiments, the foam may be sufficiently stable such that it remains substantially foam-like as it is rubbed between the hands and/or worked into the hair. In at least some embodiments, the foam of compositions according to the disclosure may remain stable for more than 5 minutes, such as more than 10 minutes, more than 20 minutes, more than 30 minutes, more than 45 minutes, more than 60 minutes, or more than 90 minutes, without a significant change in volume and/or density.

The foam compositions according to the present disclosure are generally easy to spread throughout the hair, which may improve coverage. The compositions further are able to provide style and/or shaping, and in at least in some embodiments may provide care benefits to hair, especially curly hair. The compositions may also have cosmetic attributes such as curl definition and elongation, as well as frizz control, shine, maintaining hydration of over 8 hours in some cases, ease of styling, no crunch, no build up, fast drying, and/or are not sticky.

II. Packaging Systems

The present disclosure also relates to packaging systems comprising the compositions described herein. The packaging systems comprise at least one container suitable for containing and/or applying the composition and/or transforming the compositions from a gel into a foam.

In various embodiments, a packaging system according to the present disclosure may include a dispensing container containing a composition disclosed herein. The composition contained in the dispensing container is generally transparent or semi-transparent, i.e., clear or semi-clear and in the form of a gel.

Compositions may optionally be in aerosol or non-aerosol form. If they are in aerosol form, the compositions or packaging systems may comprise at least one propellant. If they are in non-aerosol form they do not need a propellant. Instead, they can be packaged in any dispensing container that has a dispensing device configured to dispense its contents as non-aerosol foam with or without controlled amount or doses.

By way of non-limiting example, suitable dispensing container may be any type of non-aerosol foam dispenser container. Such container may including a foaming chamber having a mesh therein and/or a foam distributing nozzle, so called foam pump, pump dispenser, or pump foamer. The dispensing container may have any size or shape and may be made of any suitable material(s).

The dispensing container may be prefilled with a composition according to the disclosure. The dispensing container may also be configured to allow filling or refilling with a composition disclosed herein when the dispensing container is empty or when the composition contained therein is running low, by the user at any time or near the time of use.

III. Methods

The present disclosure also relates to methods for styling keratin fibers, e.g. human hair, using the compositions described herein. The methods generally comprise applying any of the disclosed compositions to the keratin fibers. According to the present disclosure, compositions described herein are particularly useful for styling, shaping, and/or defining curly hair.

In various exemplary embodiments, the methods may include applying a styling- or shaping-effective amount of a foam composition of the present disclosure to hair (wet, damp, or dry hair), before, during, and after styling and shaping the hair. The composition may be left on the hair for any period of time, such as a few hours or a few days, or until the next washing or rinsing of the hair.

In some embodiments, a composition of the disclosure may be applied to the hair and left on the hair, e.g. as a mousse for holding curls or other hair styles. In one embodiment, the hair is allowed to air dry after application of the composition, and is styled or shaped with no heat being applied to the hair. Styling or shaping the hair may involve the use of devices on hair such as a brush, a comb or running the fingers of the hand through the hair. Optionally, styling or shaping can be done using twisting out techniques. In this situation, the composition disclosed herein may be applied to the hair before or during performing twist out, where the hair is twist and then let go to create curls or other hair styles.

In another embodiment, the methods may include styling or shaping the hair with a heating device before, during, or after the compositions are applied to the hair.

Additionally, the compositions provide a variety of desirable sensory benefits to the hair, for example, smoothness without weigh-down, detangling, anti-frizz, softness, moisture, and/or anti-shrinkage. As such, the compositions are useful in styling or shaping hair while also caring for hair, conditioning hair, and/or imparting one or more of smoothness, detangling, and/or frizz control to hair. Accordingly, the instant disclosure encompasses methods for treating hair with the compositions of the instant disclosure. For example, the methods may also include applying the compositions to curly hair to boost volume or hold existing curl styles, as well as providing additional sensory benefits to the hair, e.g. conditioning.

As described herein, due to the transformative texture or transformative nature of the gel compositions, the gel is transformed into a foam when a shear stress is applied to the gel. Thus, the disclosure also relate to methods of transforming gel compositions described herein into foams by applying shear stress to the gel.

Having described the many embodiments of the present invention in detail, it will be apparent that modifications and variations are possible without departing from the scope of the disclosure defined in the appended claims. Furthermore, it should be appreciated that all examples in the present disclosure, while illustrating many embodiments of the disclosure, are provided as non-limiting examples and are, therefore, not to be taken as limiting the various aspects so illustrated. It is to be understood that all definitions herein are provided for the present disclosure only.

As used herein, the terms “comprising,” “having,” and “including” (or “comprise,” “have,” and “include”) are used in their open, non-limiting sense. The phrase “consisting essentially of” limits the scope of a claim to the specified materials or steps and those that do not materially affect the basic and novel characteristics of the compositions.

In this application, the use of the singular includes the plural unless specifically stated otherwise. The singular forms “a,” “an,” “the,” and “at least one” are understood to encompass the plural as well as the singular unless the context clearly dictates otherwise. The expression “one or more” means “at least one” and thus includes individual components as well as mixtures/combinations. Likewise, the term “a salt thereof” also relates to “salts thereof.” Thus, where the disclosure refers to “an element selected from the group consisting of A, B, C, D, E, F, a salt thereof, or mixtures thereof,” it indicates that that one or more of A, B, C, D, and F may be included, one or more of a salt of A, a salt of B, a salt of C, a salt of D, a salt of E, and a salt of F may be included, or a mixture of any two of A, B, C, D, E, F, a salt of A, a salt of B, a salt of C, a salt of D, a salt of E, and a salt of F may be included.

Throughout the disclosure, if the term “a mixture thereof,” or “a combination thereof” including variants, is used, following a list of elements as shown in the following example where letters A-F represent the elements: “one or more elements selected from the group consisting of A, B, C, D, E, F, or mixtures thereof.” The term, “a mixture thereof” does not require that the mixture include all of A, B, C, D, E, and F (although all of A, B, C, D, E, and F may be included). Rather, it indicates that a mixture of any two or more of A, B, C, D, E, and F can be included. In other words, it is equivalent to the phrase “one or more elements selected from the group consisting of A, B, C, D, E, F, and a mixture of any two or more of A, B, C, D, E, and F.”

For purposes of the present disclosure, it should be noted that to provide a more concise description, some of the quantitative expressions given herein are not qualified with the term “about.” It is understood that whether the term “about” is used explicitly or not, every quantity given herein is meant to refer to the actual given value, and it is also meant to refer to the approximation to such given value that would reasonably be inferred based on the ordinary skill in the art, including approximations due to the experimental and/or measurement conditions for such given value. All ranges and amounts given herein are intended to include sub-ranges and amounts using any disclosed point as an end point. Thus, a range of “1% to 10%, such as 2% to 8%, such as 3% to 5%,” is intended to encompass ranges of “1% to 8%,” “1% to 5%,” “2% to 10%,” and so on. All numbers, amounts, ranges, etc., are intended to be modified by the term “about,” whether or not so expressly stated. Similarly, a range given of “about 1% to 10%” is intended to have the term “about” modifying both the 1% and the 10% endpoints. The term “about” is used herein to indicate a difference of up to +/−10% from the stated number, such as +/−9%, +/−8%, +/−7%, +/−6%, +/−5%, +/−4%, +/−3%, +/−2%, or +/−1%. Likewise, all endpoints of ranges are understood to be individually disclosed, such that, for example, a range of 1:2 to 2:1 is understood to disclose a ratio of both 1:2 and 2:1.

“Active material” as used herein with respect to the percent amount of an ingredient or raw material, refers to 100% activity of the ingredient or raw material.

All amounts given herein are relative to the amount of active material, unless otherwise indicated.

All percentages, parts and ratios herein are based upon the total weight of the compositions of the present disclosure, unless otherwise indicated.

As used herein, the terms “applying a composition onto keratin materials” and “applying a composition onto hair” and variations of these phrases are intended to mean contacting the keratin materials including hair and skin, with at least one of the compositions of the disclosure, in any manner. It may also mean contacting the keratin materials in an effective amount.

Unless otherwise indicated, all percentages herein are by weight, relative to the weight of the total composition.

As used herein, the term “conditioning” means imparting to hair fibers at least one property chosen from combability, moisture-retentivity, luster, shine, and softness. The state of conditioning can be evaluated by any means known in the art, such as, for example, measuring, and comparing, the ease of combability of the treated hair and of the untreated hair in terms of combing work, and consumer perception.

As used herein, the term “efflux” refers to the rate at which a gel composition according to the present disclosure drains out of a cup used for measuring the viscosity of the gel composition. “Efflux time” refers to the time that the composition takes to completely drain out of the cup under the effect of gravity. Typically, higher viscosity corresponds to a longer efflux time.

As used herein, the terms “non-sulfate-based” or “non-sulfate” anionic surfactants means that the surfactant does not comprise a sulfate group.

As used herein, the term “organic” means a material that is produced substantially without or essentially without the use of synthetic materials.

The terms “substantially without” or “essentially without” as used herein means the specific material may be used in a manufacturing process in small amounts that do not materially affect the basic and novel characteristics of the compositions according to the disclosure. The terms may also mean that the specific material is not used in a manufacturing process but may still be present in a raw material that is included in the composition.

As used herein, the term “salts” refers to throughout the disclosure may include salts having a counter-ion such as an alkali metal, alkaline earth metal, or ammonium counterion. This list of counterions, however, is non-limiting.

As used herein, the term “substantially free” or “essentially free” as used herein means the specific material may be present in small amounts that do not materially affect the basic and novel characteristics of the compositions according to the disclosure. For instance, there may be less than 2% by weight of a specific material added to a composition, based on the total weight of the compositions (provided that an amount of less than 2% by weight does not materially affect the basic and novel characteristics of the compositions according to the disclosure. Similarly, the compositions may include less than 2%, less than 1.5%, less than 1%, less than 0.5%, less than 0.1%, less than 0.05%, or less than 0.01%, or none of the specified material. Furthermore, all components that are positively set forth in the instant disclosure may be negatively excluded from the claims, e.g., a claimed composition may be “free,” “essentially free” (or “substantially free”) of one or more components that are positively set forth in the instant disclosure. The term “substantially free” or “essentially free” as used herein may also mean that the specific material is not added to the composition but may still be present in a raw material that is included in the composition.

As used herein, the term “sulfate-based surfactant” as used herein, also means “sulfate-containing surfactant.” Thus, the term “essentially free of sulfate-based surfactant” also means “essentially free of sulfate-containing surfactant.”

As used herein, the term “surfactants,” as well as any specifically-identified surfactants, includes salts of the surfactants even if not explicitly stated.

As used herein, the term “surfactant system” refers to a combination of different surfactants. For example, the term “anionic surfactant system” refers to one anionic surfactant or a combination of different anionic surfactants, and the term “nonionic surfactant system” refers to one nonionic surfactant or a combination of different nonionic surfactants.

As used herein, the term “synthetic” means a material that is not of natural origin. The term “natural” and “naturally-sourced” means a material of natural origin, such as derived from plants, which also cannot be subsequently chemically or physically modified. “Plant-based” means that the material came from a plant.

As used herein, the term “transformative texture” or “transformative nature” refers to the texture or nature of compositions according to the disclosure that can be changed from a gel to a foam when a shear stress is applied to the gel.

As used herein, the term “treat” (and its grammatical variations) refers to the application of the compositions of the present disclosure onto the surface of keratin materials, such as hair.

Unless otherwise expressly stated, it is in no way intended that any method set forth herein be construed as requiring that its steps be performed in a specific order. Accordingly, where a method claim does not expressly recite an order to be followed by its steps or it is not specifically stated in the claims or descriptions that the steps are to be limited to a specific order, it is no way intended that any particular order be inferred.

### EXAMPLES

The following examples are intended to be non-limiting and explanatory in nature only. In the Examples, amounts are expressed in percentage by weight (wt %) of active materials, relative to the total weight of the composition.

### Example 1—Formulation of a Gel Composition

The following inventive gel composition was prepared according to the formulation set forth in Table 1 below.

TABLE 1

| | Inventive |
|---|---|
| INCI Name | Composition 1 |
| HYDROXYPROPYL GUAR | 0.25 |
| XANTHAN GUM | 0.9 |
| VP/VA COPOLYMER | 3.0 |
| DECYL GLUCOSIDE | 1.59 |
| SODIUM C14-16 OLEFIN SULFONATE | 0.76 |
| PEG-14 DIMETHICONE | 1.9 |
| SORBITOL (HYDROGENTATED STARCH | 0.7 |
| HYDROLYSATE) | |
| GLYCERIN | 4.0 |
| ADDITIVES (fragrance, preservatives, | 2.0 |
| thickeners, vitamins, pH adjusters, antioxidants) | |
| WATER | QS |

Inventive Composition 1 was prepared as follows. First, mix the solvents and the hydroxypropyl guar and xanthan gum until all hydroxypropyl guar and xanthan gum were dispersed in the solvents, then, add the rest ingredients except citric acid one at a time and mixing during each addition. After mixing well, check the pH of the composition and adjust the pH to about 4.5 with citric acid.

Inventive Composition 1 was semi-clear to clear and looked like a gel. The gel can transform to stable, semi-transparent foam when a shear stress is applied to the gel. The pH of Inventive Composition 1 was about 3.5 to about 4.5 at 25° C. The viscosity of Inventive Composition 1 was measured by a viscometer Rheomat 180 model at 25° C. with Spindle #2. The measurement was performed after 30 seconds of rotation of the spindle in the composition at a shear rate of 200 rpm. The viscosity can range from 200 cps to 500 cps.

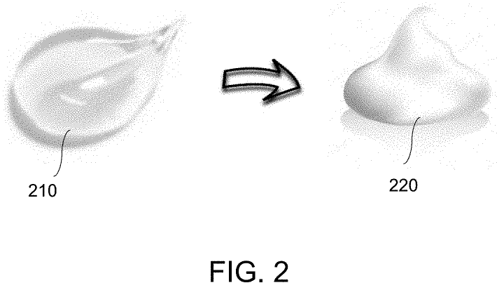

Inventive Composition 1 had a gel-like texture 210 as illustrated in FIG. 2. Under a shear stress, gel 210 transformed to foam 220 shown in FIG. 2. The density of the foam, when evaluated by the volume percentage of the total bubbles, was greater than 40% v/v.

Inventive Composition 1 in the form of a gel may be stable for at least one year, under ambient conditions. Thus, Inventive Composition 1 in the form of a gel has a Period After Opening (PAO) characteristic of at least one year, which means it may remain stable and safe for human use at least one year after the package containing the composition has first been opened.

### Example 2—Mechanism of the Texture Transformation from Get to Foam

A study of the mechanism of texture transformation from gel to foam was performed on Inventive Composition 1, prepared according to the formulation of Table 1, in comparison with five commercially available compositions (C1, C2, C3, C4, and C5), of which composition C1 is a shampoo, composition C2 is a spray hair gel, composition C3 is a traditional hair styling gel, composition C4 is a hair styling foaming liquid, and composition C5 is a hair milk (lotion). The flow property of each sample was characterized using a rheometer (DHR-3, TA instruments, New Castle, Del., USA) with 40 mm parallel plates. The gap between the parallel plates was 1 mm. All tests were conducted at 25° C. and atmospheric pressure. The sample was subjected to shear ramp starting from 0.1 1/s to 1000 1/s within a 300 seconds period. The time interval between data points is 1 second. The shear stress response was recorded for every data point.

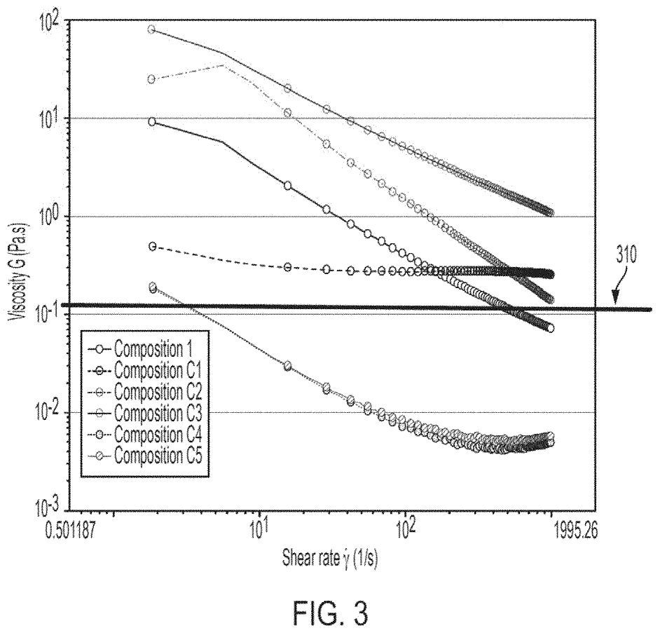

FIG. 3 illustrates the study result. As shown in FIG. 3 the viscosity of Inventive Composition 1 decreased when shear stress was applied to the gel of Inventive Composition 1, which means that Inventive Composition 1 was shear thinning. When the shear stress generated by the pumping system was sufficient, the gel of Inventive Composition 1 transformed into a foam. In contrast, although Compositions C1 and C3 were shear thinning gels, when shear stress was applied to them, the viscosities did not decrease to a level that allowed them to transform into foam. Composition C2 displayed Newtonian fluid behavior, which means its viscosity had little change when the applied shear rate changed. Even under high shear rate generated by the pumping system, the viscosity of Composition C2 remained in a level that did not allow it to foam. Compositions 4 and 5 could transform into foam; however, they were thin liquids and were not gels within the temperature range when the shear stress was applied.

### Example 3—Foam Study

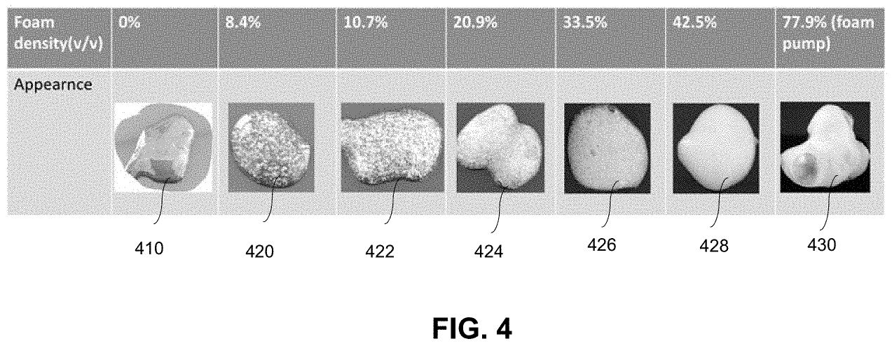

A foam study was conduct to evaluate the foaming property of Inventive Composition 1. The result is illustrated in FIG. 4. In this study, a set of samples of Inventive Composition 1, in about the same amount, were filled into a bottle and be shaken at different speeds or durations by hand, respectively. The shaking simulated a shearing action and introduced air into the samples. Another sample of Inventive Composition 1 was filled into a foam pump and dispensed therefrom. As shown in FIG. 4, before being shaken or being dispensed, Inventive Composition 1 was in the form of gel 410. After being shaken by hand at different speeds or durations, bubbles in liquid carrier thereof were formed from the gel with foam densities of 8.4%, 10.7%, 20.9%, 33.5%, and 42.5% shown in samples 420, 422, 424, 426, and 428, respectively. Foam sample 430 was obtained by dispensing gel 410 from the foam pump and had a density of about 78%. All densities described herein are in the unit of v/v, corresponding to the volume of the bubbles in a sample to the total volume of the bubbles and the liquid carrier thereof in the sample. The foam of sample 430 was very stable, with a change of density from about 78% v/v to about 75% v/v after 90 minutes.

The study demonstrated that even by hand shaking, Inventive Composition 1 could transform from gel to foam, reaching a density over about 40% v/v. In addition, the foam was very stable with little change to its volume or density, even after 90 minutes.

### Example 4—Sensory Properties Evaluation

A comparative study was conducted to evaluate the performance of Inventive Composition 1 in comparison with the commercially available compositions C2 and C5, as described in Example 2. Composition C2 was in the form of a gel and could not transform into foam. Composition C5 was not a gel but was able to foam. In this test, similar amounts of foam or gel of Inventive Composition 1, and compositions C2 and C5 were respectively applied to the same hair (African American Curly Panel) that have curl types VI and VII.

The attributes were assessed and the data showed that, compared to compositions C2 and C5, Inventive Composition 1 received high ratings in curl definition, frizz control, shine, hydration over 8 hours, ease of styling, no crunch, no build up, dries faster, not sticky product, and elongation. Inventive Composition 1 was also found to perform better in providing hair care and styling benefits than composition C2.

It will be apparent to those skilled in the art that various modifications and variations can be made in the compositions and methods according to the disclosure without departing from the spirit or scope of the disclosure. Thus, it is intended that the disclosure cover such modifications and variations and their equivalents.

1-42. (canceled)

43. A gel composition comprising:
(a) at least one nonionic guar polymer;
(b) at least one polysaccharide other than the at least one nonionic guar polymer;
(c) at least one nonionic film forming polymer;
(d) at least one nonionic surfactant;
(e) at least one anionic surfactant;
(f) at least one silicone compound; and
(g) at least one polyol;
wherein the gel composition has a transformative texture that can be transformed into a foam when shear stress is applied.

44. The gel composition according to claim 43, wherein the at least one nonionic guar polymer comprises a nonionic hydroxyalkyl-modified guar polymer.

45. The gel composition according to claim 43, wherein the at least one nonionic guar polymer is present in an amount ranging from about 0.01% to about 2% by weight, relative to the total weight of the composition.

46. The gel composition according to claim 43, wherein the at least one polysaccharide other than the at least one nonionic guar polymer is chosen from gums, celluloses, starches, or mixtures thereof.

47. The gel composition according to claim 43, wherein the at least one polysaccharide other than the at least one nonionic guar polymer is present in an amount ranging from about 0.01% to about 2% by weight, relative to the total weight of the composition.

48. The gel composition according to claim 43, wherein the weight ratio of the at least one polysaccharide other than the at least one nonionic guar polymer to the at least one nonionic guar polymer ranges from about 1:0.01 to about 1:1.

49. The gel composition according to claim 43, wherein the at least one nonionic film forming polymer is chosen from vinylpyrrolidone homopolymers, copolymers of vinylpyrrolidone and of vinyl acetate, polyalkyloxazolines, vinyl acetate homopolymers, copolymers of vinyl acetate and of acrylic ester, copolymers of vinyl acetate and of ethylene, copolymers of vinyl acetate and of maleic ester, copolymers of polyethylene and of maleic anhydride, alkyl acrylate homopolymers, alkyl methacrylate homopolymers, copolymers of acrylonitrile and of a non-ionic monomer, or mixtures thereof.

50. The gel composition according to claim 43, wherein the at least one nonionic film forming polymer is present in an amount ranging from about 0.1% to about 15% by weight, relative to the total weight of the composition.

51. The gel composition according to claim 43, wherein the at least one nonionic surfactant is chosen from alkyl or polyalkyl esters of poly(ethylene oxide), alkyl or polyalkyl ethers of poly(ethylene oxide), optionally polyoxyethylenated alkyl or polyalkyl esters of sorbitan, optionally polyoxyethylenated alkyl or polyalkyl ethers of sorbitan, alkyl or polyalkyl glycosides polyglycosides, alkyl or polyalkyl esters of sucrose, optionally polyoxyethylenated alkyl or polyalkyl esters of glycerol, salts thereof, or mixtures thereof.

52. The gel composition according to claim 43, wherein the at least one nonionic surfactant comprises an alkylpolyglucoside, a salt thereof, or mixtures thereof.

53. The gel composition according to claim 43, wherein the at least one nonionic surfactant is present in an amount ranging from about 0.01% to about 10% by weight, relative to the total weight of the composition.

54. The gel composition according to claim 43, wherein the at least one anionic surfactant is chosen from non-sulfate anionic surfactants comprising alkyl sulfonates, alkyl sulfosuccinates, alkyl sulfoacetates, acyl isethionates, alkoxylated monoacids, acyl amino acids, acyl glycinates, acyl glutamates, acyl sarcosinates, salts thereof, or mixtures thereof.

55. The gel composition according to claim 43, wherein the at least one anionic surfactant is present in an amount ranging from about 0.01% to about 6% by weight, relative to the total weight of the composition.

56. The gel composition according to claim 43, wherein the at least one silicone compound is chosen from dimethicone copolyols.

57. The gel composition according to claim 43, wherein the at least one silicone compound is present in an amount ranging from about 0.1% to about 10% by weight, relative to the total weight of the composition.

58. The gel composition according to claim 43, wherein the at least one polyol comprises at least one sugar alcohol and at least one alkane polyol, wherein the sugar alcohol is present in the composition in an amount ranging from about 0.05% to about 5% by weight, relative to the total weight of the composition, and the alkane polyol is present in an amount ranging from about 0.1% to about 15% by weight, relative to the total weight of the composition.

59. A gel composition comprising:
(a) from about 0.01% to about 2% by weight of at least one nonionic guar polymer;
(b) from about 0.01% to about 2% by weight of at least one polysaccharide other than the at least one nonionic guar polymer;
(c) from about 0.1% to about 15% by weight of at least one nonionic film forming polymer;
(d) from about 0.01% to about 10% by weight of at least one nonionic surfactant;
(e) from about 0.01% to about 6% by weight of at least one anionic surfactant;
(f) from about 0.1% to about 10% by weight of at least one silicone compound;
(g) at least one polyol chosen from sugar alcohols; and
(h) at least one polyol chosen from alkane polyols;
wherein the weight ratio of the at least one polysaccharide other than the at least one nonionic guar polymer to the at least one nonionic guar polymer ranges from about 1:0.01 to about 1:1.

60. The gel composition according to claim 59, wherein the gel composition has a transformative texture that can be transformed into a foam when a shear stress is applied by mechanical means.

61. A packaging system comprising a dispensing container containing a gel composition comprising:
(a) at least one nonionic guar polymer;
(b) at least one polysaccharide other than the at least one nonionic guar polymer;
(c) at least one nonionic film forming polymer;
(d) at least one nonionic surfactant;
(e) at least one anionic surfactant;
(f) at least one silicone compound; and
(g) at least one polyol
wherein the dispensing container comprises a non-aerosol dispensing device configured to provide shear stress to the gel composition sufficient to transform the form of the gel composition into a foam.

62. A method for caring for, styling, and/or shaping hair, the method comprising:
(i) applying shear stress to a gel composition comprising:
(a) at least one nonionic guar polymer;
(b) at least one polysaccharide other than the at least one nonionic guar polymer;
(c) at least one nonionic film forming polymer;
(d) at least one nonionic surfactant;
(e) at least one anionic surfactant;
(f) at least one silicone compound; and
(g) at least one polyol
wherein after the shear stress is applied to the gel composition, the composition is in the form of a foam; and
(ii) applying the foam to the hair.
